(19) United States
(12) Patent Application Publication
(10) Pub. No.: US 2014/0213951 A1
(43) Pub. Date: Jul. 31, 2014

(54) ROBOTIC GAIT REHABILITATION TRAINING SYSTEM WITH ORTHOPEDIC LOWER BODY EXOSKELETON FOR TORQUE TRANSFER TO CONTROL ROTATION OF PELVIS DURING GAIT

(71) Applicants: Maciej Pietrusisnki, Cambridge, MA (US); Constantinos Mavroidis, Arlington, MA (US); Paolo Bonato, Somerville, MA (US); Ozer Unluhisarcikli, Allston, MA (US); Iahn Cajigas, Cambridge, MA (US); Brian Weinberg, San Diego, CA (US)

(72) Inventors: Maciej Pietrusisnki, Cambridge, MA (US); Constantinos Mavroidis, Arlington, MA (US); Paolo Bonato, Somerville, MA (US); Ozer Unluhisarcikli, Allston, MA (US); Iahn Cajigas, Cambridge, MA (US); Brian Weinberg, San Diego, CA (US)

(73) Assignees: SPAULDING REHABILITATION HOSPITAL CORPORATION, Boston, MA (US); NORTHEASTERN UNIVERSITY, Boston, MA (US)

(21) Appl. No.: 14/129,206

(22) PCT Filed: Jun. 25, 2012
(86) PCT No.: PCT/US12/44019

§ 371 Date: Mar. 27, 2014

Related U.S. Application Data
(60) Provisional application No. 61/500,797, filed on Jun. 24, 2011.

(51) Int. Cl. A61F 5/01

(52) U.S. Cl. CPC A61F 5/0102
USPC 602/23

(57) ABSTRACT

A robotic gait rehabilitation (RGR) training system is provided to address secondary gait deviations such as hip-hiking. An actuation assembly follows the natural motions of a user's pelvis, while applying corrective moments to pelvic obliquity. A human-robot interface (HRI), in the form of a lower body exoskeleton, is provided to improve the transfer of corrective moments to the pelvis. The system includes an impedance control system incorporating backdrivability that is able to modulate the forces applied onto the body depending on the patient's efforts. Various protocols for use of the system are provided.

### CROSS REFERENCE TO RELATED APPLICATIONS

This application claims priority under 35 U.S.C. §119(e) of U.S. Provisional Patent Application No. 61/500,797, filed on Jun. 24, 2011, the disclosure of which is incorporated by reference herein.

### STATEMENT REGARDING FEDERALLY SPONSORED RESEARCH OR DEVELOPMENT

This invention was made under National Science Foundation NSF Grant No. 0803622. The Government may have certain rights in the invention.

### BACKGROUND OF THE INVENTION

Many people suffer from diseases or injuries that affect their ability to walk. For example, strokes can result in various primary gait deviations, such as knee hyperextension during stance or limited knee flexion during swing. Secondary gait deviations, which result from compensating for primary gait deviations, can also occur. Hip-hiking is the most common secondary gait deviation. Many of these people can benefit from rehabilitation such as physical therapy to improve or regain their walking ability.

### SUMMARY OF THE INVENTION

A robotic gait rehabilitation (RGR) training system is provided that facilitates robotic gait retraining of patients, particularly patients experiencing secondary gait deviations such as hip-hiking. The RGR training system includes an actuation system that uses force fields applied to the pelvis of a patient to correct secondary gait deviations in pelvic motion. The system also includes a human-machine or human-robot interface that includes a lower body exoskeleton. The human-robot interface improves torque transmission to the pelvic region. The RGR training system implements impedance control-based human-robot interface modalities that allow a patient to interact with the system in ways that mimic the interaction with a therapist walking along side and manually assisting movement of the patient. Several protocols for use of the RGR training system are provided. The protocols are useful for rehabilitation of patients experiencing secondary gait deviations such as hip-hiking and for research with healthy subjects.

### DESCRIPTION OF THE DRAWINGS

The invention will be more fully understood from the following detailed description taken in conjunction with the accompanying drawings:

FIG. 1 is an illustration of the human gait cycle;

FIG. 2 is a schematic illustration of the frontal, sagittal and transverse planes through a human body;

FIG. 3A illustrates pelvic drop in normal gait;

FIG. 3B illustrates anterior tilt in normal gait;

FIG. 3C illustrates rotation in normal gait;

FIG. 4 illustrates hip-hiking;

FIG. 5 illustrates circumduction;

FIG. 6 is an illustration of one embodiment of a robotic gait rehabilitation (RGR) training system employed in conjunction with a user ambulating on a treadmill;

FIG. 7 is a schematic illustration of application of a moment applied onto the pelvis about the weight-supporting hip joint with a single actuator;

FIG. 8 is a schematic illustration of an embodiment of an actuation system of a RGR training system;

FIG. 9A is an illustration of a basic linear actuator;

FIG. 9B is a cross-sectional view of the linear actuator of FIG. 9A;

FIG. 10 illustrates a further embodiment of a mounting assembly for mounting the linear actuation assembly to a frame;

FIG. 11 is a partial exploded view of the mounting assembly of FIG. 10;

FIG. 12 is a partial view of a revolute joint of the mounting assembly of FIG. 10;

FIG. 13 is a front view of a human-robot interface (HRI);

FIG. 14 is a schematic view of a pelvic brace illustrating correspondence with a user's hip joints;

FIG. 15A is an illustration of a pelvic brace in a closed position;

FIG. 15B is an illustration of the pelvic brace in FIG. 15A in an open position;

FIG. 16A is an illustration of a right side of the HRI of FIG. 13 illustrating degrees of freedom;

FIG. 16B is an illustration of a right side of the HRI of FIG. 13 illustrating adjustments;

FIG. 17 is an illustration of an embodiment of a hip revolute joint with potentiometer for flexion-extension angle measurement;

FIG. 18A is an illustration of an embodiment of a knee joint with adjustable frontal plane angle and rotary potentiometer for knee flexion/extension measurement, at one extreme position;

FIG. 18B is an illustration of the knee joint of FIG. 18A at another extreme position;

FIG. 19 illustrates an embodiment of a control system;

FIG. 20 is a schematic illustration of a simple impedance control architecture;

FIG. 21 is a schematic of a simple model of an actuator thrust rod;

FIG. 22 is a schematic illustration of an actuator shaft and force control law;

FIG. 23 is a schematic illustration of a physical implementation of Equation 14;

FIG. 24 is a schematic illustration of the calculation of the obliquity angle;

FIG. 25 is a schematic diagram of an inner current loop in one embodiment of a servo amplifier;

FIG. 26 is a schematic diagram of a PD controller acting on the obliquity error;

FIG. 27 is a schematic diagram of the PD gain block of FIG. 26;

FIG. 28 illustrates a conceptual diagram and synchronization algorithm diagram;

FIG. 29 is a schematic illustration of one embodiment of an overall system architecture;

FIG. 30 is a graph of two consecutive gait cycles illustrating synchronization of the control system with a gait;

FIG. 31 is an illustration of a first protocol for use with an RGR training system;

FIG. 32 is an illustration of a second protocol for use with an RGR training system;

FIG. 33 is an illustration of a third protocol for use with an RGR training system;

FIG. 34 is an illustration of a fourth protocol for use with an RGR training system;

FIG. 35 is a schematic illustration of an embodiment of a 2 DOF system;

FIG. 36 is a schematic illustration of a further embodiment of a 2 DOF system;

FIG. 37 is a schematic illustration of a still further embodiment of a 2 DOF system;

FIG. 38 is a schematic illustration of a still further embodiment of a 2 DOF system;

FIG. 39 illustrates a further embodiment of an RGR training system;

FIG. 40 illustrates a linear actuator assembly of the embodiment of FIG. 39;

FIG. 41 illustrates an actuator mount from the embodiment of FIG. 39;

FIG. 42 illustrates a closed linkage mechanism of the embodiment of FIG. 39;

FIG. 43 is a schematic top view illustration of the embodiment of FIG. 39;

FIG. 44 schematically illustrates pelvic obliquity and rotation angles for the embodiment of FIG. 39;

FIG. 45 illustrates a force of resolved into component magnitudes and unit vectors for the embodiment of FIG. 39;

FIG. 46 is an isometric view of a frame for use with the embodiment of FIG. 39;

FIG. 47 is a side view of the frame of FIG. 46, further illustrating a treadmill and wheelchair access;

FIG. 48 illustrates an embodiment of attachment of the embodiment of FIG. 39 to the frame;

FIG. 49 illustrates an embodiment of a handlebar height adjustment mechanism; and

FIG. 50 illustrates an embodiment of a handlebar tilt adjustment mechanism.

### DETAILED DESCRIPTION OF THE INVENTION

The disclosure of U.S. Provisional Patent Application No. 61/500,797, filed on Jun. 24, 2011, is incorporated by reference herein.

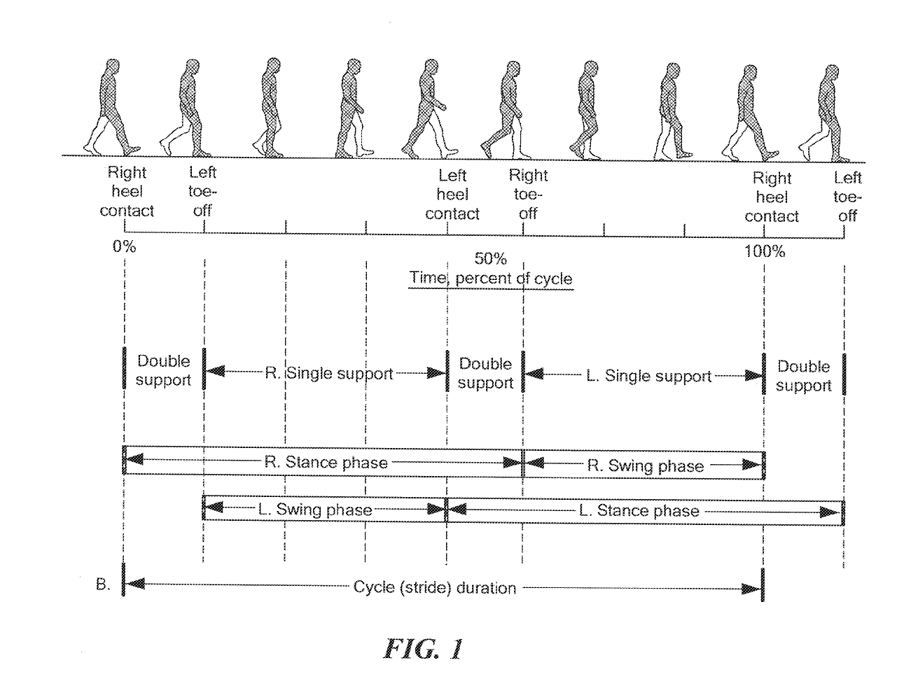

Human gait is comprised of strides, which are the intervals between two consecutive heel strikes. See FIG. 1. Gait markers, such as toe-off, are used to identify the phases of gait, the swing phase and the stance phase. The stance phase lasts approximately 60% of the gait cycle, while the swing phase takes up the remaining 40%. Both limbs are in contact with the ground for about 10% of the cycle, which is referred to as double limb support.

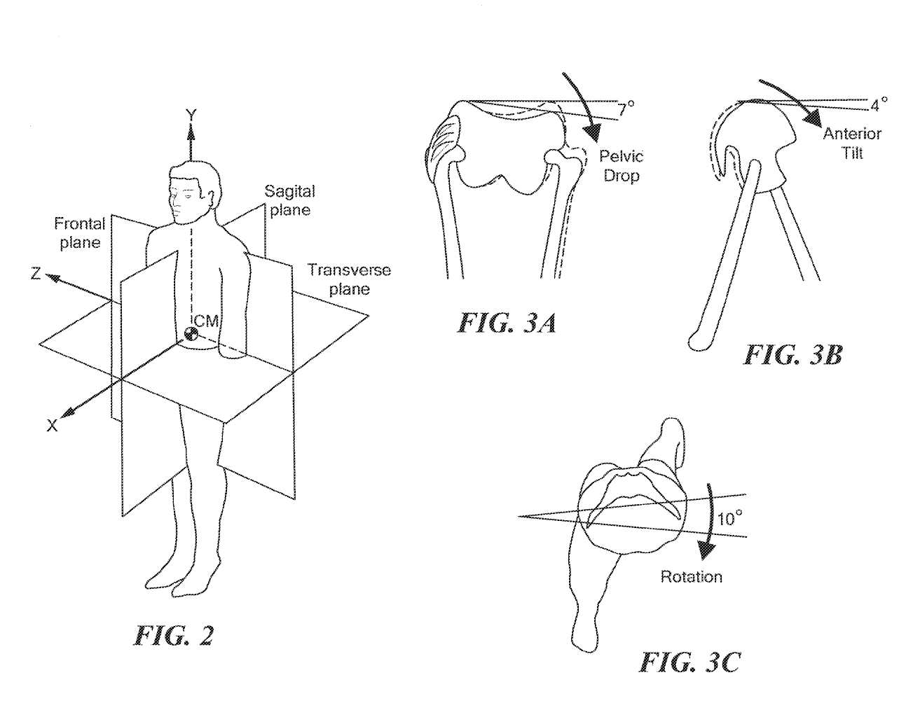

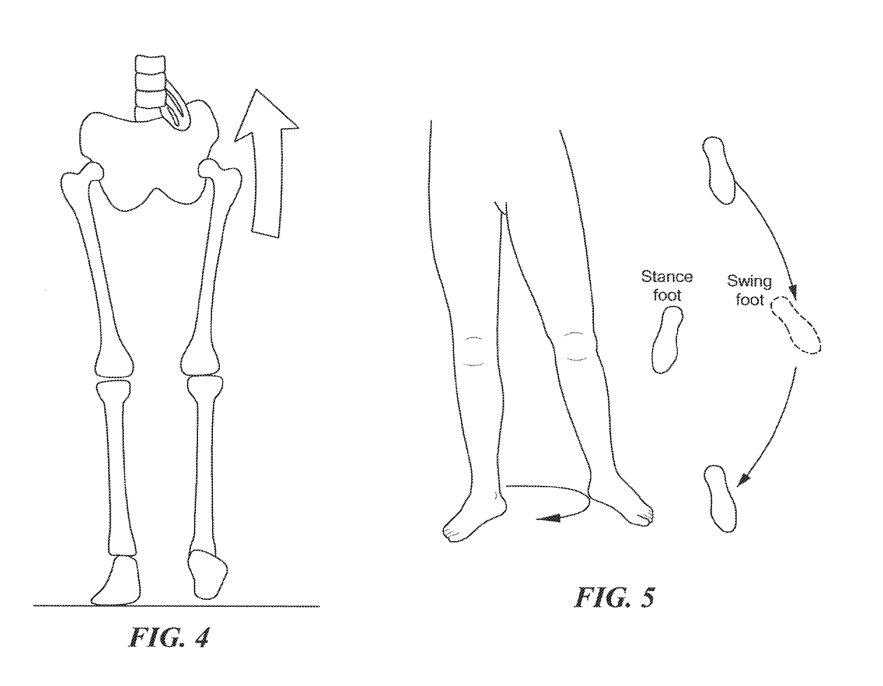

During normal gait, the pelvis rotates in three planes: frontal, sagittal and transverse. See FIG. 2. Rotation of the pelvis in the frontal plane is termed obliquity, rotation in the sagittal plane is termed pelvic tilt, and rotation in the transverse plane is termed pelvic rotation. During single limb support, these rotations happen about the supporting limb's hip joint. Pelvic drop, anterior tilt and rotation are normal events which occur during normal gait in obliquity, pelvic tilt and pelvic rotation respectively. See FIGS. 3A, 3B, and 3C.

The most common primary gait deviation in patients post-stroke is stiff-legged gait. This gait deviation often results in the patient employing secondary gait deviations that involve motor control of the pelvis. Stiff legged gait is associated with hip hiking or circumduction. Hip hiking is an exaggerated elevation of the pelvis on the contralateral side (i.e. hemiparetic side) to allow toe clearance during swing. See FIG. 4. Circumduction is an exaggerated rotation of the pelvis in combination with an exaggerated hip abduction. See FIG. 5. Abnormal control of pelvic obliquity and rotation of the pelvis are the most common secondary gait deviations observed in post-stroke patients. A patient employs these secondary gait deviations in order to assist in foot clearance when either hip flexion or knee flexion are inadequate.

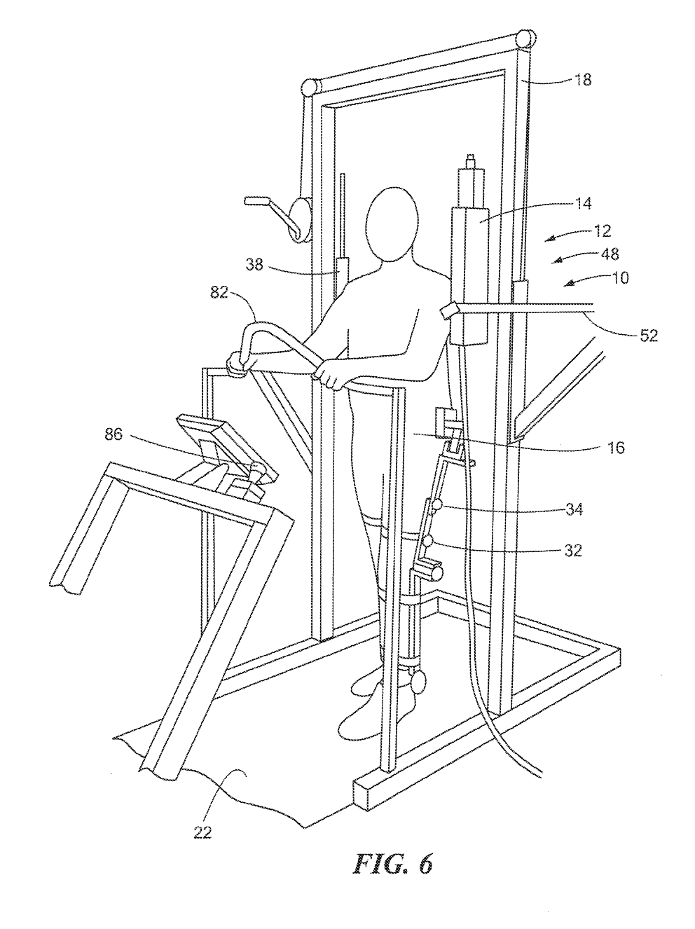

To administer gait retraining therapy, a robotic gait rehabilitation (RGR) training system is provided that generates force fields around a user's pelvis while the user ambulates on a treadmill. The RGR training system in particular targets hip-hiking. One embodiment of an RGR training system 10 is illustrated in FIG. 6. The RGR training system includes an actuation system 12, which follows the natural motions of the subject's pelvis, while applying corrective moments to pelvic obliquity as determined by a control system. The actuation system includes a linear actuator 14 that is attached at one end to a pelvic brace 16 worn by the user and is also attached to a stable frame 18 that remains stationary and is placed over the treadmill 22. The actuation system operates in conjunction with an impedance control system incorporating backdrivability. The control system, described further below, is able to modulate the forces applied onto the body depending on the patient's efforts. A human-robot interface (HRI) 32, in the form of a lower body exoskeleton 34, is provided to improve the transfer of corrective moments to the pelvis. The HRI employs the waist, thighs, shanks and feet of the user to effectively and reliably impart significant forces onto the user's lower body and alter the orientation of the pelvis in the frontal plane (pelvic obliquity).

In the embodiment of FIG. 6, the RGR training system controls one degree of freedom (DOF) in the motion of the pelvis: obliquity. The remaining two rotational DOFs (pelvic rotation and pelvic tilt) and three translational DOFs are non-actuated (except for the ground reaction force on the foot of the non-actuated side).

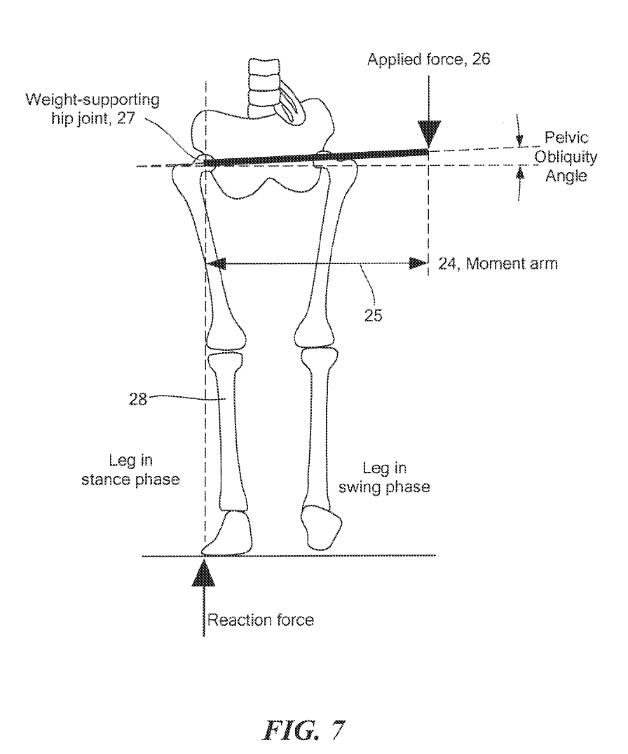

Referring more particularly to FIGS. 6-8, the RGR training system employs a single actuator 14 on one side of the body. The center of rotation of the pelvis shifts with respect to the center of mass of the body throughout the gait cycle. Despite this, a single force with an appropriately chosen line of action can exert a fully controllable moment onto the pelvis in the frontal plane. See FIG. 7. The moment arm 24 consists of a line segment 25 perpendicular to the line of action of the applied force 26 and spanning between it and the hip joint 27 of the supporting leg 28 (this does not hold true during double support stance).

The control system (described further below) activates the force field only when the leg on the affected side (the hemiparetic leg) is believed to be in swing. This makes it possible to use only one actuator to generate a well-defined moment around the pelvis in the frontal plane, with a vertical reaction force at the support leg, which is equal in magnitude to the applied force generated by the actuator. In one embodiment, the RGR training system uses a synchronization algorithm, discussed further below, which produces an estimate of the subject's location in their own gait cycle, to control the timing of the actuator.

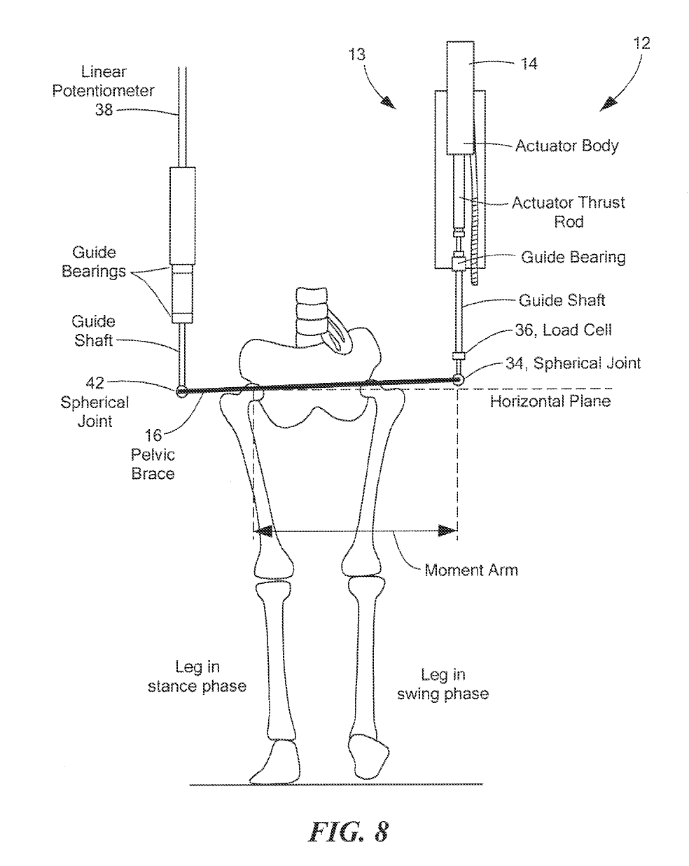

Referring to FIG. 8, the actuation system 12 includes a linear actuator assembly 13 attached at a spherical joint 34 to the pelvic brace on one side of the user to transfer forces from the actuator to the brace. The linear actuator assembly applies a corrective moment to the pelvis by acting along the line of applied force shown in FIG. 7. The linear actuator assembly includes a linear actuator 14 and a tension-compression load cell 36 disposed in alignment with the linear actuator. The load cell, in communication with the control system, provides force feedback for control and performance evaluation. A linear potentiometer assembly 38 provides vertical position feedback on the side opposite the linear actuator assembly 13. The linear potentiometer 40 is also attached at a spherical joint 42 to the pelvic brace 16. The linear actuator 14 and the linear potentiometer 40 are fixed to the frame 18 but can follow the motion of the body in the horizontal plane. Guide bearings and guide shafts are included as needed to ensure vertical linear motion of the linear actuator assembly and the linear potentiometer assembly. In FIGS. 6 and 8, actuation is illustrated as provided on the left side of the body, assuming that is the patient's weaker side. It will be appreciated that the system can be reconfigured to provide actuation on the right side of the body, for example, by moving the above described components to the opposite side.

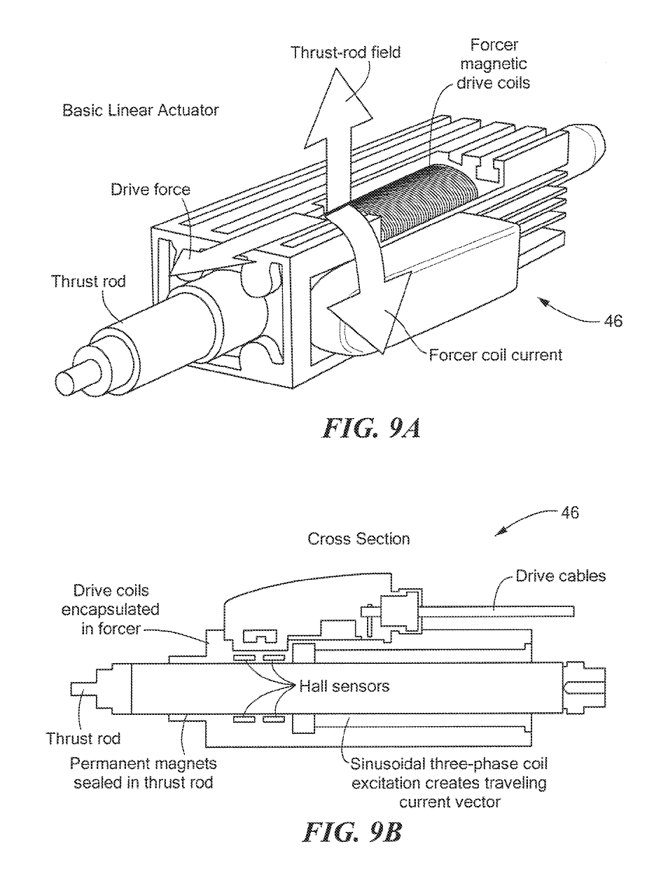

In one embodiment, force generation is suitably achieved via a servo-tube actuator 46, which is a good source of force and lends itself well to impedance control. A suitable servo-tube actuator 46 is Model STA2508, from Copley Controls Inc. (Canton, Mass., USA), which incorporates a direct-drive electromagnetic linear motor, with windings in the actuator housing, and permanent rare-earth magnets in the movable thrust-rod (see FIGS. 9A and 9B). The thrust rod is extended by a precision shaft, which is guided by two linear ball bearings. Hall-effect sensors provide actuator position feedback by sensing the series of permanent magnets in the thrust rod (see FIG. 9B). It will be appreciated, however, that other forms and types of force generation can be used.

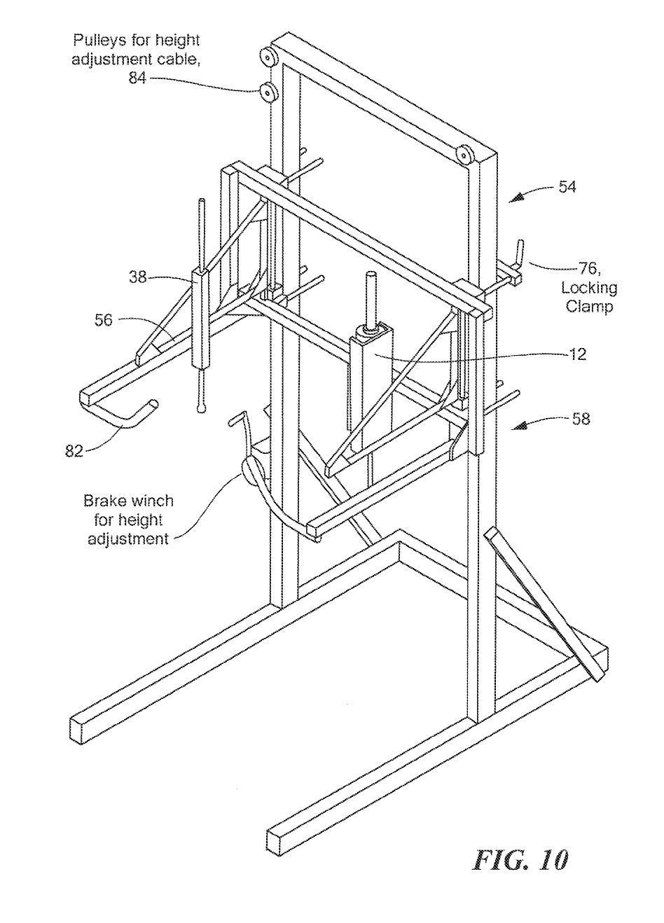

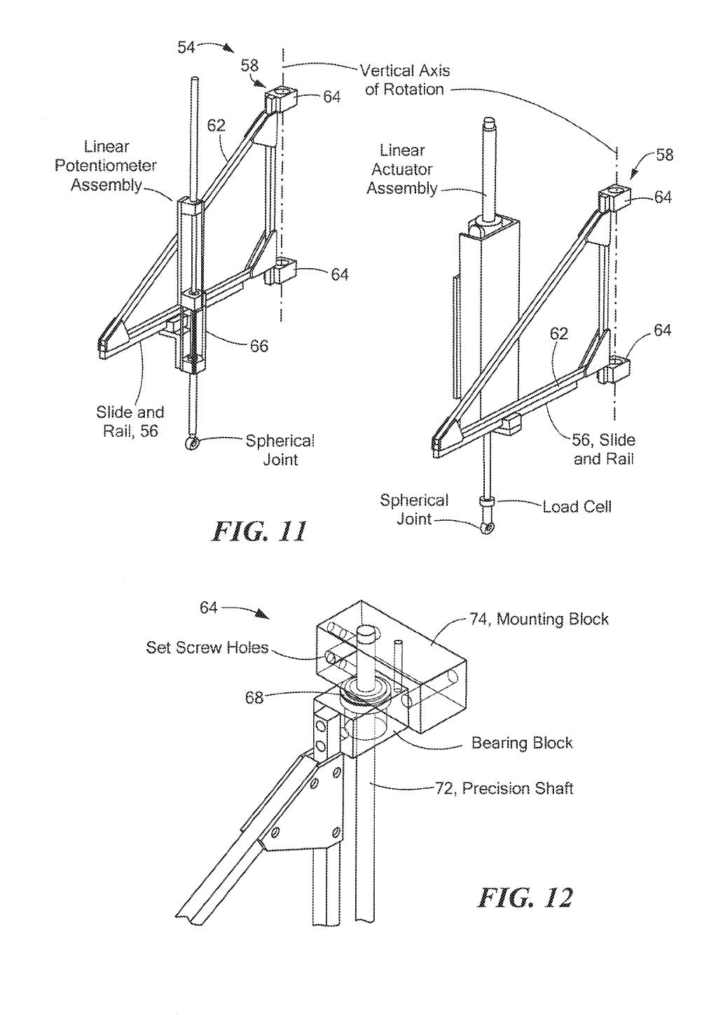

The actuation system 12 of the RGR training system 10 is suspended over the treadmill 22 with a suitable frame 18. In the embodiment of FIG. 6, a mounting assembly 48 including two sets of linear guides 52 is provided on each side. In another embodiment, a mounting assembly 54 includes a linear guide 56 and a rotary joint 58 on each side. See FIGS. 10-12. Both embodiments enable the actuation system to follow the user in the horizontal plane with little friction, while resisting forces in the vertical direction. The embodiment of FIGS. 10-12 shifts the vertical component of the frame back behind the user, giving easier access to the user's legs.

Referring more particularly to FIGS. 10-12, the linear actuator assembly 12 and the linear potentiometer assembly 38 are each mounted on triangular frame subassemblies 62. A revolute joint 64 about the vertical axis and a prismatic joint 66 in the horizontal plane provide unconstrained motion in the horizontal plane while constraining motion in the vertical direction. For example, each triangular frame subassembly is supported with two tapered roller bearings 68 at the revolute joints, which are located concentrically on precision shafts 72. Two opposing bearings on each side support axial loads in either direction, and radial loads. Mounting blocks 74 are locked to the precision shaft, for example, via set screws, and maintain a fixed distance between the bearings. The mounting blocks can be affixed in any suitable manner to the frame, for example, with locking clamps 76 on the frame uprights.

The frame 18 of the system provides a rigid support for the linear actuation system so that forces can be safely and accurately applied onto a user's pelvis. The frame also provides mounting for body weight support and provides support for upper body, for example, via a handle bar 82. In some embodiments, the frame can also provide unrestricted access for physical therapist from the side to either leg of the subject.

The frame includes structural elements, joined in any suitable manner, for example, with threaded fasteners or by welding. The frame is preferably wide enough for a wheel chair to enter the frame from the rear. The frame can include any suitable height adjustment mechanism 84, such as a pulley and brake winch assembly on the frame uprights, to accommodate users of different heights. The frame elements are fabricated from a suitably strong material, for example, rectangular cross-sectional steel tubing. The frame can be made modular to be easily installed on site.

A handlebar or handlebars 82 can be grasped by the user during gait training. The height and tilt of the handlebars can be adjustable. The handlebars can also be adjustable fore and aft. Quick release adjustment mechanisms can be used, for example, quick release clamps. When the clamps are tightened, the structural rigidity of the frame is enhanced. The adjustment mechanisms can be simplified, for example, with the use of knobs, indexing plungers, and quick-release clamps, to allow the adjustments to be performed by a single person without the need for tools. An emergency stop 86 can also be included, for example, on one of the handlebars.

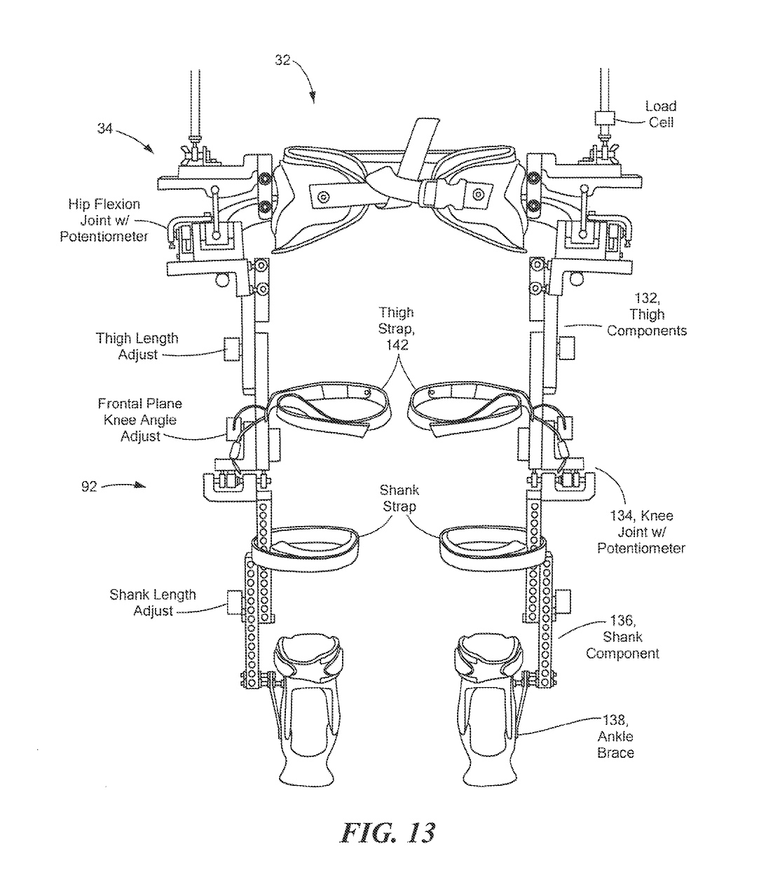

An embodiment of the human-machine or human-robot interface (HRI) 32 is illustrated in FIGS. 13-18B. Due to the presence of soft tissue and the lack of prominent skeletal features in the pelvic region, transfer of torques to alter the orientation of the pelvis and measurement of its orientation in space are challenging. For example, the soft tissue around the pelvic region undergoes significant deformation when torques are applied to a belt tightened around the waist or pelvis. Also, the applied torques can cause migration, or slipping, of a belt relative to the skin around the pelvic region.

The HRI addresses these challenges by providing an exoskeleton 34 that improves torque transmission to the pelvic region by employing not only adherence to the pelvic region, but also adherence to the thighs, shanks and feet of a patient. Migration of the pelvic brace 16 is substantially eliminated due to the use of the patient's feet, which are positioned transversely to the action of the applied forces of interest, for anchoring the brace to the body. Alteration of the patient's gait is minimal due to the design of the exoskeleton's hip joints, which allow for hip flexion/extension and abduction/adduction, while still transferring forces through the hip joints to the pelvis. This interface maximizes the effectiveness of force transfer to the pelvis, while minimizing time and effort necessary to don and doff the system.

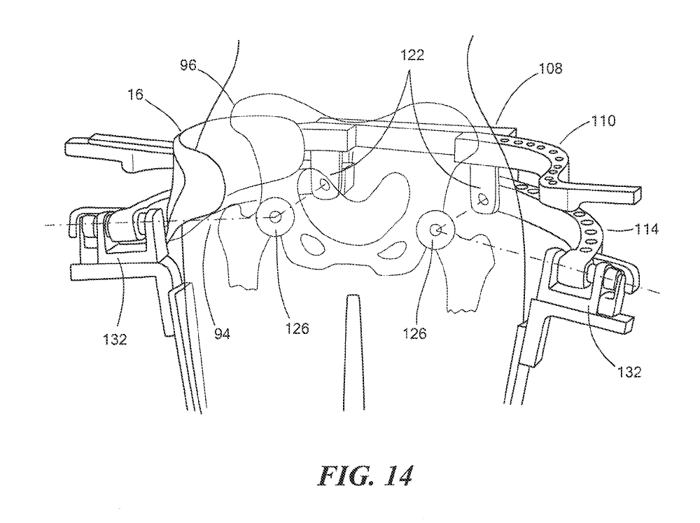

One embodiment of a human-robot interface (HRI) 32, shown in FIG. 13, includes an exoskeleton 34 having three subassemblies: the pelvic brace 16 and two leg braces 92 that together span all the major joints in the lower body: ankle, knee and hip. The pelvic brace wraps around the user's waist, above the greater trochanter 94 of the femur and below the iliac crest 96 of the pelvis, as shown in FIG. 14. Each leg brace attaches to a leg of the user with structural elements extending along the outside.

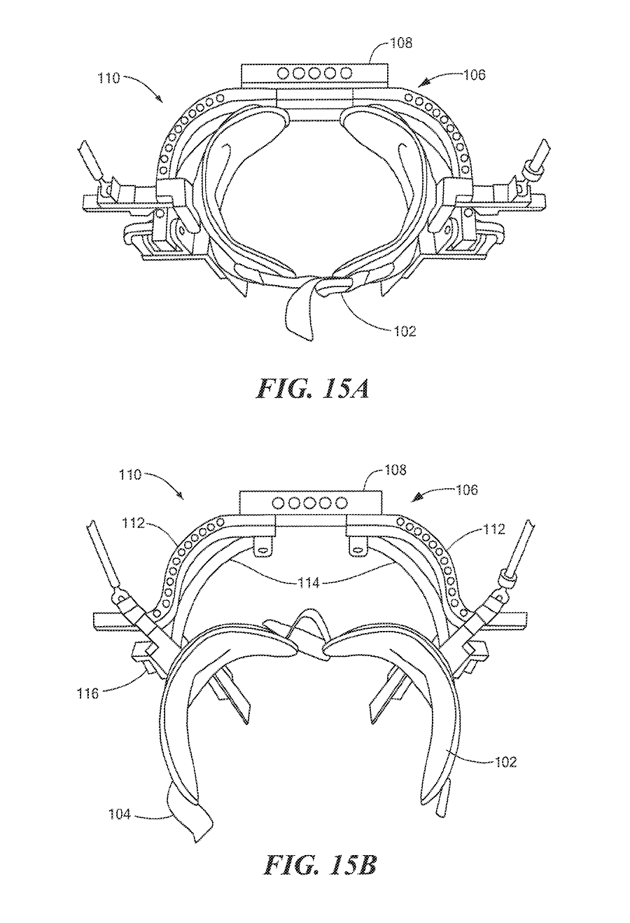

The pelvic brace includes a shell 102, formed in two halves, that wraps around and fastens to a user's waist, thereby locating the HRI with respect to the body in the horizontal plane. See FIGS. 15A and 15B. Fore-aft position adjustment and lateral position adjustment can be accomplished via, for example, straps 104. In this manner, the HRI can closely track motion of the pelvis while not being directly affected by motion of the upper torso. The pelvic brace also includes a rigid pelvic frame assembly 106 that includes a center piece 108 at the back and a curved side section 110 on each side. Each side section includes an upper arm 112 and a lower abductor 114. The shell attaches to the upper arm via a movable bracket 116 on each side.

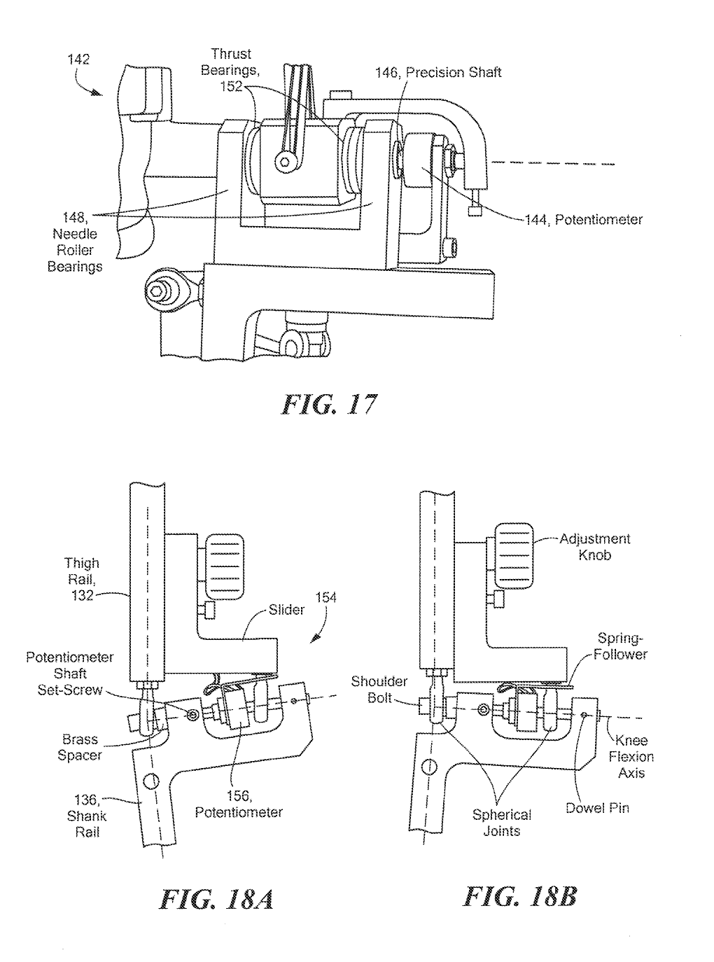

The pelvic brace 16 is coupled to the two leg braces 92 and to the actuation system via four rotational joints. See FIG. 14. The joints are double-supported with two roller bearings and two thrust bearings each. (See also FIG. 17.) The horizontal center piece 108 in the back locates the two abduction/adduction joints 122 coincidentally with the patients' hip joints 126 in the frontal plane. The two curved arms 112 attach to the center piece 108 and transfer forces generated by the actuation system to the user's pelvis. The two abductors 114 rotate about the abduction/adduction joints 122 and transfer forces and moments to the flexion/extension joints 132 located in the frontal plane. Thus, the four rotational joints together produce two remote center of rotation joints, which coincide with the patients' hip joints 126. See FIG. 14. By linking the exoskeleton's leg braces with the pelvic brace using rotational joints which are co-located or substantially co-located with those of the user, it becomes possible to employ the majority of the lower body to transfer moments to the pelvis.

Each leg brace includes a thigh component 132, a knee joint 134, a shank component 136, and an ankle brace 138. The thigh component is attachable to a user's thigh in any suitable manner, such as with a thigh strap 142. Similarly, the shank component is attachable to a user's shank in any suitable manner, such as with a shank strap 144.

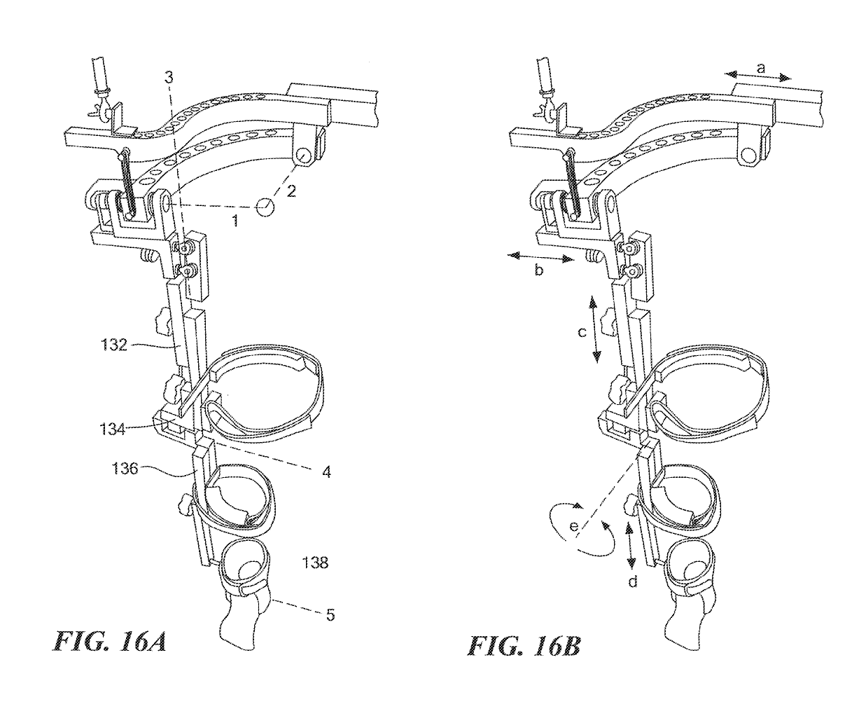

Referring to FIGS. 16A and 16B, the HRI has various free DOFs and adjustments to lower body size and shape. Each side of the HRI explicitly accommodates 5 DOFs: 1) hip flexion/extension, 2) hip abduction/adduction, 3) hip internal/external rotation, 4) knee flexion/extension, and 5) ankle plantarflexion/dorsiflexion. See FIG. 16A. Ankle inversion/eversion is accommodated implicitly through shifting and play in the fit of the ankle brace 138 inside the shoe. Through proper HRI adjustment to the user, all the DOFs can nearly coincide with the user's joint axes, except for hip internal/external rotation axes, which are shifted several inches away from the anatomical axes.

Referring to FIG. 16B, adjustments can be made at a) the hip joint span, b) the pelvis width, c) the thigh length, d) the shank length, and e) the knee frontal plane angle. The thigh component can be adjustable in the length, for example, by incorporating two rails with an adjustment mechanism therebetween. Similarly the shank component can be adjustable in the length, for example, by incorporating two rails with an adjustment mechanism therebetween. The knee joint can include an angle adjustment mechanism. The center piece of the pelvic frame assembly can be adjustably attached to the side sections. The location of the abduction/adduction joints can be shifted laterally to accommodate a range of hip joint spans.

Rotational joint assemblies 142, such as that shown in FIG. 17, that link the rigid pelvic frame assembly to the leg braces, can include sensors, such as a potentiometer 144, for flexion-extension angle measurement. In this embodiment, a precision shaft 146 is double supported by a pair of needle pin roller bearings 148 and thrust bearings 152. Other suitable rotational joint configurations can be used.

The knee joint can include an adjustment mechanism 154 of the frontal plane knee angle. An embodiment, illustrated in FIGS. 18A and 18B, provides space for a knee flexion/extension measuring sensor, such as potentiometer 156, while maintaining a compact configuration. The knee joint is shown in FIGS. 18A and 18B, at two extremes of rotation. The potentiometer's rotor is aligned with the centers of rotations of the two spherical joints, and rotates with the shank component (held with a set-screw), while the body of the potentiometer rotates with the thigh component due to a music wire spring. Though external/internal rotation of the leg is not accounted for in the HMI with a rotational joint, the flexibility of the knee brace can allow for a significant range of motion in that DOF.

The HRI can include load-carrying components that can withstand forces resulting form the structure supporting the full weight of a 244 lb (110 kg) user, which corresponds to a US male in the 99^{th} percentile, with a safety factor of 2. The structural components can be fabricated from, for example, high strength aluminum alloy 7075.

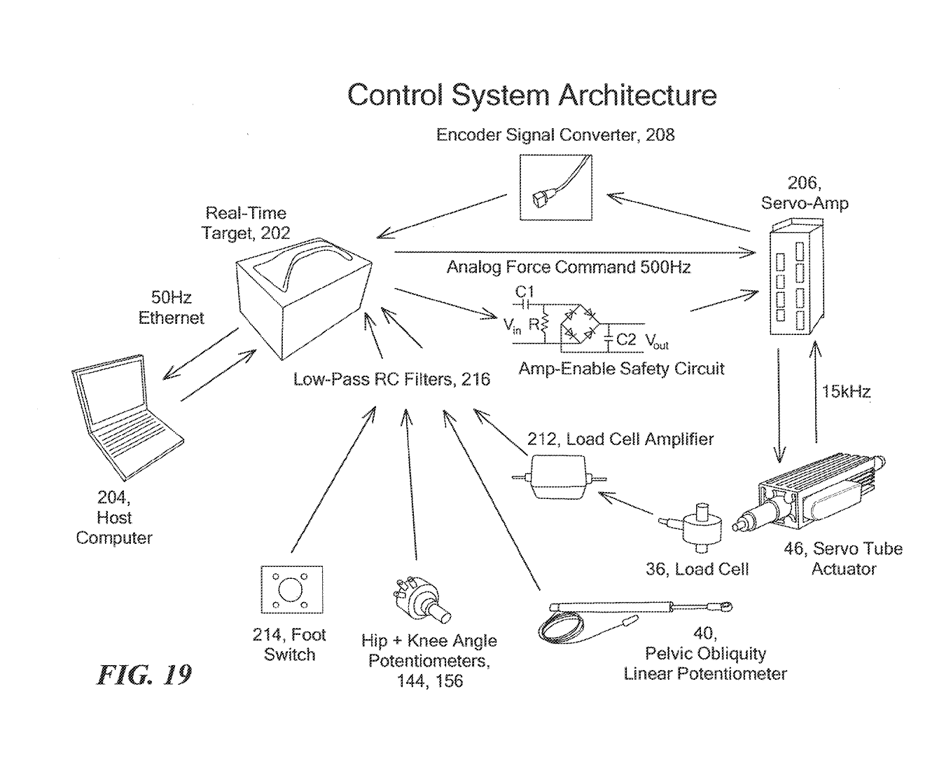

Hardware components of one embodiment of the control system 200 are illustrated in an exploded layout in FIG. 19. The system includes a controller 202 (a real time target), which may be, for example, a dedicated PC, and a user interface 204 which may be another PC (a host), running any suitable operating system, such as Windows OS, for use by, for example, a therapist. The real time environment allows for controller operation that is not interrupted by non-critical tasks, as may happen in non-deterministic operating systems such as Windows. The servo tube linear actuator 46 is in communication with a servo amplifier 206 that communicates with the real-time target via an encoder signal converter 208. The real-time target provides force commands to the servo-amplifier. Similarly, signals from the load cell 36, via a load cell amplifier 212, are transmitted to the real-time target. Signals from the pelvic obliquity linear potentiometer 40, the hip and knee angle potentiometers 144, 156 and foot switch 214 are similarly transmitted to the real-time target. Low-pass RC filters 216 are also provided, discussed further below.

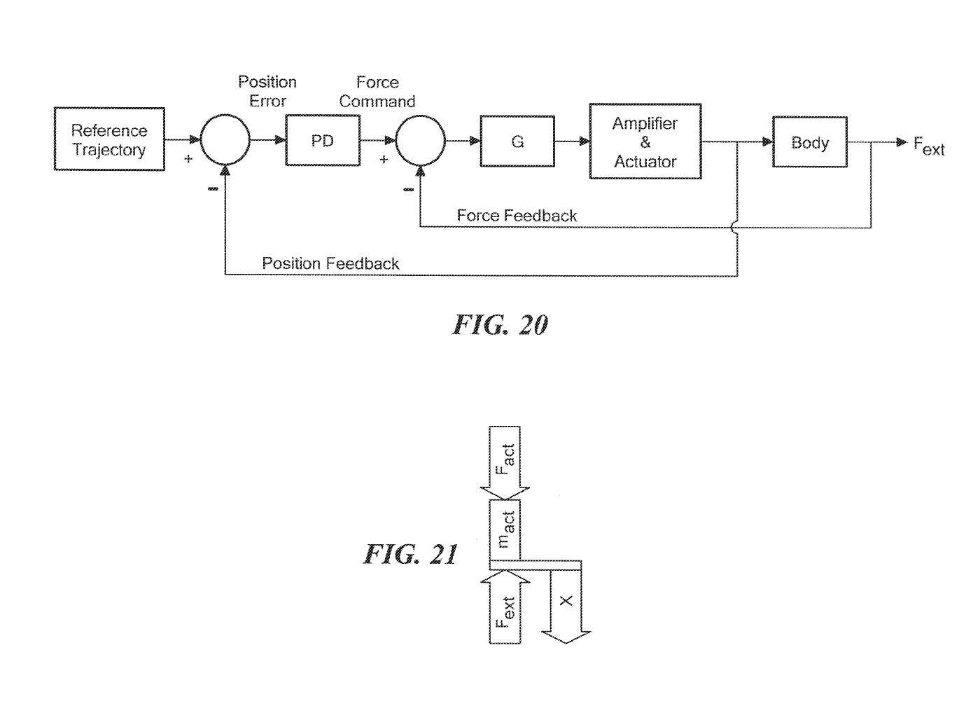

An noted above, the RGR training system employs an impedance control system. Impedance control in this context refers to the control of the end-point impedance of a robot or an actuator. Impedance control architecture comprises an inner unity feedback force loop, and an outer unity feedback position loop. The main task of the force loop is to increase backdrivability of the actuator. In that sense, force feedback moves any actuator closer to an ideal source of force. The outer position loop sets the relationship between the position of the end-effector, and the force it exerts. In control theory, this can usually be accomplished with a PD controller, where the proportional term represents virtual spring stiffness, and the derivative term acts like a virtual damper. A simple schematic of an impedance controller is shown in FIG. 20. The proportional and derivative gains (PD) produce a force command that is executed by the force loop with gain G. The system's interaction force with the environment (Fₑₓₜ) is measured with a load cell.

FIG. 21 illustrates a simple model of an actuator's thrust rod. Neglecting friction, the actuator's thrust rod can be represented as a mass m undergoing displacement x due to forces F_{act} applied by the actuator's electromagnetic field, and Fₑₓₜ, or external force, applied by the environment.

m_{act}\ddot{x}=F_{act}−Fₑₓₜ (1)

The equation describing a simple closed loop control law is:

F_{act}=G(F_{ref}−Fₑₓₜ) (2)

These two equations combined give the following equation:

m_{act}\ddot{x}=G(F_{ref}−Fₑₓₜ)−Fₑₓₜ (3)

And the transfer function is:

$$X = \frac{{GF}_{ref} - ( G + 1 ) {F}_{ext}}{{m}_{act} {s}^{2}} \qquad ( 4 )$$

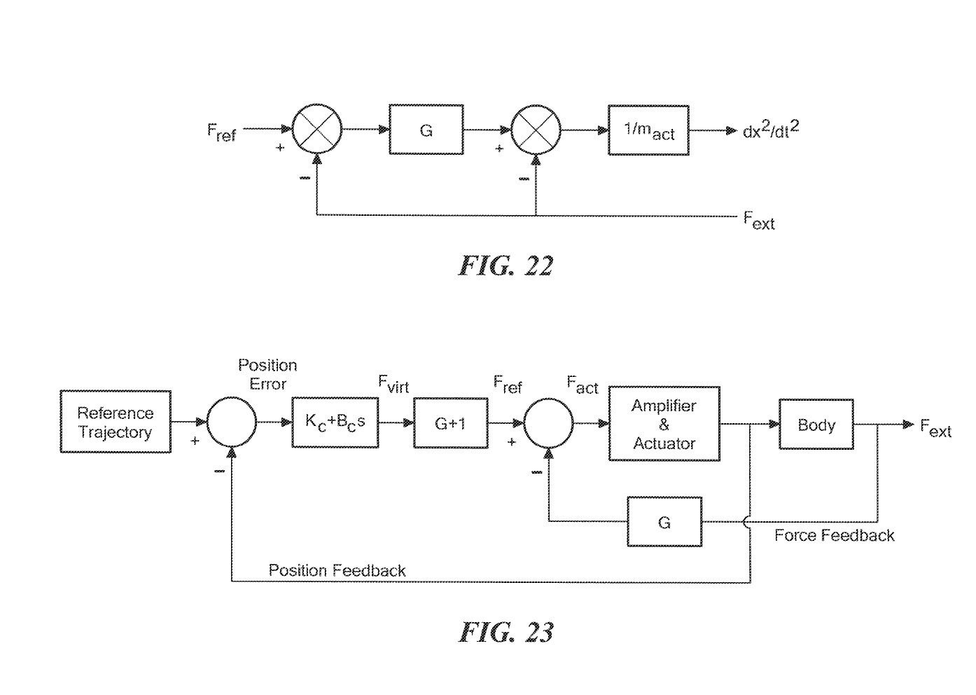

This can be represented by the block diagram in FIG. 22, illustrating the actuator shaft and force control law.

The immovable mass (body) with stiffness and damping, with Fₑₓₜ being the interaction force between the body and the actuator, can be represented by the first order equation:

Fₑₓₜ=Bₑ\dot{x}+Kₑx (5)

### and its Laplace is:

$$X = \frac{{F}_{ext}}{{B}_{e} s + {K}_{e}} \qquad ( 6 )$$

The actuator transfer function (Equation 4) is equated with the body's transfer function (Equation 6) to describe the actuator-body interaction:

$$\frac{{F}_{ext}}{{F}_{ref}} = \frac{( {B}_{e} s + {K}_{e} ) \frac{G}{( G + 1 )}}{\frac{{m}_{act}}{( G + 1 )} {s}^{2} + {B}_{e} s + {K}_{e}} \qquad ( 7 )$$

where m_{act}/(G+1) is the apparent inertia as experienced by the environment. Therefore, the effect of force feedback is the reduction of the apparent actuator inertia by a factor of G+1.

The impedance controller derivation for controlling the actuator's end point impedance in the RGR training system can be derived as follows, referring to FIG. 21.

The equation describing the dynamics is:

m_{act}\ddot{x}=F_{act}−Fₑₓₜ (8)

where the force generated by the actuator (F_{act}) onto the thrust rod is:

F_{act}=m_{act}\ddot{x}+Fₑₓₜ (9)

The desired end-point impedance of the actuator thrust rod can be represented by the following equation:

Fₑₓₜ=M_{c}(\ddot{x})+B_{c}(\dot{x}₀−\dot{x})+K_{c}(x₀−x) (10)

where M_{c} is the actuator's apparent mass (inertia), B_{c} is controller derivative gain (damping) and K_{c} is controller proportional gain (stiffness).

The desired acceleration of the actuator thrust rod is:

$$\ddot{x} = \frac{1}{{M}_{c}} [ {K}_{c} ( {x}_{0} - x ) + {B}_{c} ( {\dot{x}}_{o} - \dot{x} ) - {F}_{ext} \qquad ( 11 )$$

Now substitute the desired acceleration into the actuator force equation:

$$\begin{matrix}{F}_{act} = \frac{{m}_{act}}{{M}_{c}} [ {K}_{c} ( {x}_{0} - x ) + {B}_{c} ( {\dot{x}}_{o} - \dot{x} ) - {F}_{ext} ] + {F}_{ext} & ( 12 ) \\ {F}_{act} = \frac{{m}_{act}}{{M}_{c}} [ {K}_{c} ( {x}_{0} - x ) + {B}_{c} ( {\dot{x}}_{o} - \dot{x} ) ] + {F}_{ext} [ 1 - \frac{{m}_{act}}{{M}_{c}} ] & ( 13 )\end{matrix}$$

Equation (13) above describes the impedance controller. F_{act} is the force command sent to the servo-amplifier. The inertia of the thrust rod mass, m_{act}, should be as low as possible. In practice, the degree to which this apparent inertia can be reduced by use of force feedback is limited. The desired mass M_{c} is equated to the lowest possible apparent inertia of the thrust rod: M_{c}=m_{act}/(G+1) and the force controller gain G is selected to be highest possible, while still providing appropriate stability margin. After the substitution, the equation describing force commanded to the actuator F_{act} is:

F_{act}=(G+1)[K_{c}(x₀−x)+B_{c}(\dot{x}₀−\dot{x})]−(G)Fₑₓₜ (14)

The above equation lists the constituents of the force command F_{act}, which is sent into the servo amplifier, to be executed by the actuator. This can be represented by the diagram in FIG. 23.

The output of the PD controller, which acts on the position error, can be called the virtual force, Fᵥᵢᵣₜ. It is the output of the virtual spring and virtual damper, K_{c} and B_{c} respectively.

Force controller gains are often limited to single digits. At such low gain values, the steady state error can be very significant. For example, using the control law of equation (2) and a proportional gain G=1, the resulting force output Fₑₓₜ is only 50% of the reference F_{ref}. The impedance controller from FIG. 23 takes this effect into account, magnifying the PD controller's output by (G+1) to cancel the following-error resulting from the control law and low gain value. Due to the force feedback's dependence on the environment, tuning is often performed manually.

As noted above, in one embodiment, the servo tube actuator is equipped with hall-effect sensors, which are used by the servo amplifier to generate an emulated differential quadrature encoder signal (position). The differential encoder position signal from the servo amplifier is converted to single ended using a incremental encoder adapter. The encoder signal is acquired by a data acquisition (DAQ) card, counting both rising and falling edges of the incremental encoder signal (X4 encoding). The net number of counted edges is polled by the controller at 500 Hz and converted to position with knowledge of encoder's resolution (12.5 microns).

The linear potentiometer's signal is low-pass anti-alias filtered (RC 480 Hz cutoff), and acquired by the DAQ at 2 kHz. Pelvic obliquity angle is computed as shown in FIG. 17, at the control loop's operating rate (500 Hz).

The degree to which the actuator system can actually display the specified endpoint impedances depends largely on the extent of backdrivability of the actuator. The higher the backdrivability, the better the system can display the commanded forces. Therefore, proper implementation of force feedback is advantageous for implementation of impedance control.

The signal from the load cell is amplified by an in-line amplifier. An analog anti-aliasing low pass RC filter set with an appropriate cutoff frequency, for example, 480 Hz, can improve the signal quality. This suggests that these attenuated signal components were aliases of higher frequency noise (above 480 Hz). A 4^{th} order inverse Chebyshev filter, for example, with 30 Hz cutoff and a 60 dB attenuation level, conditions the signal further.

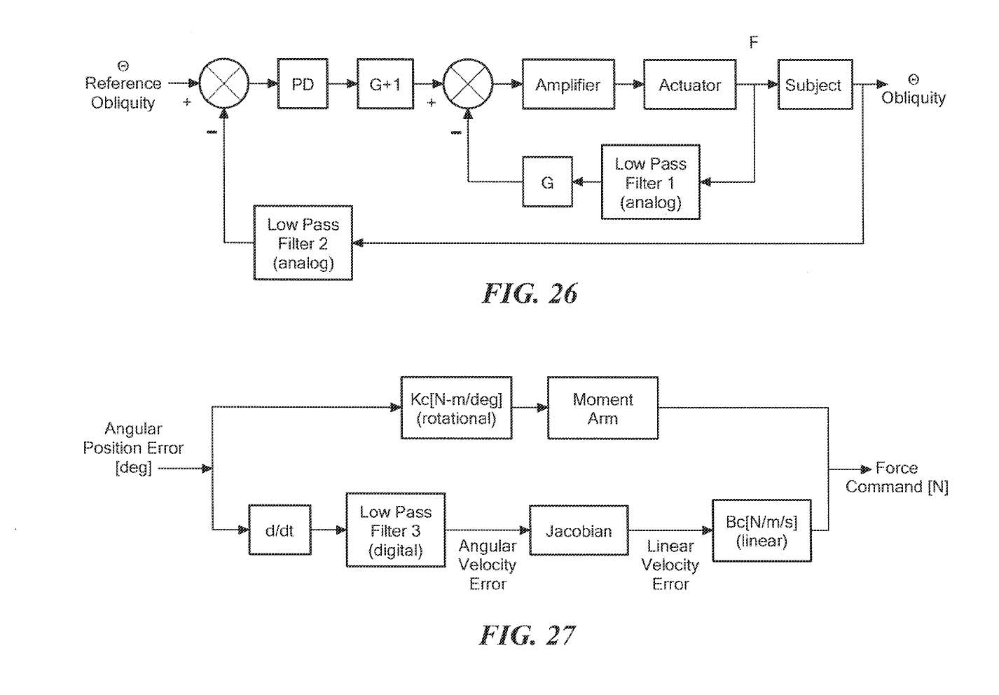

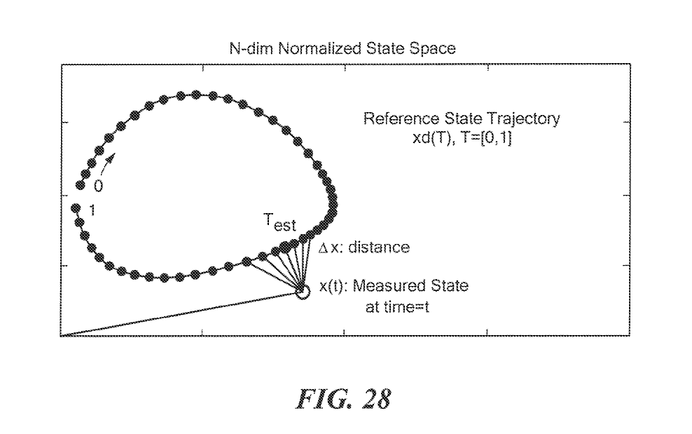

Referring again to FIG. 19, the digital servo amplifier can be programmed to operate in three different modes: position, velocity or force. In the force-mode, the servo amplifier does not use a direct measure of force, but it does monitor the current consumed by the actuator, and a proportional-integral (PI) controller adjusts the voltage sent to the actuator in order to coax the requested current draw. A block diagram of the amplifier's internal control loop is illustrated in FIG. 26. An automatic tuning procedure performed by the servo amplifier sets the current loop gains, for example to Cₚ=454 and Cᵢ=88.

As discussed above, the RGR training system applies a moment to the pelvis in the frontal plane, to affect the pelvic obliquity angle. This task requires measurement of the pelvic obliquity angle at all times throughout the gait cycle, as well as measurement of the moment or force exerted onto the user by the RGR training system.

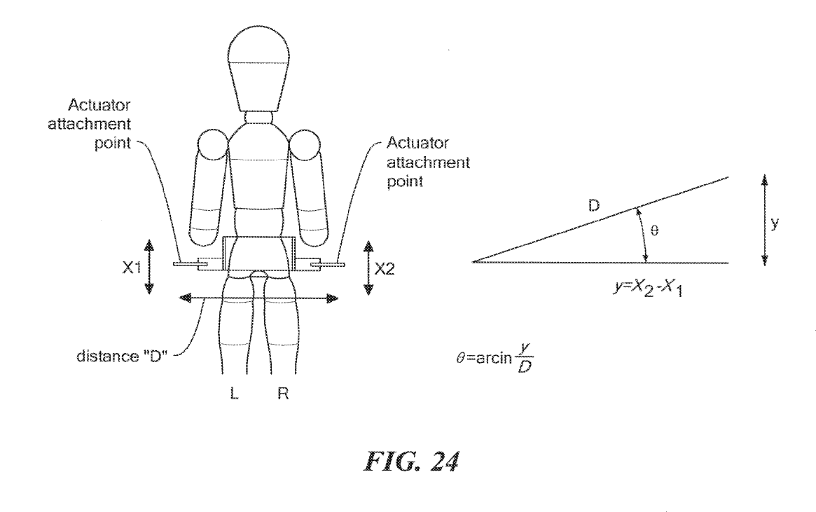

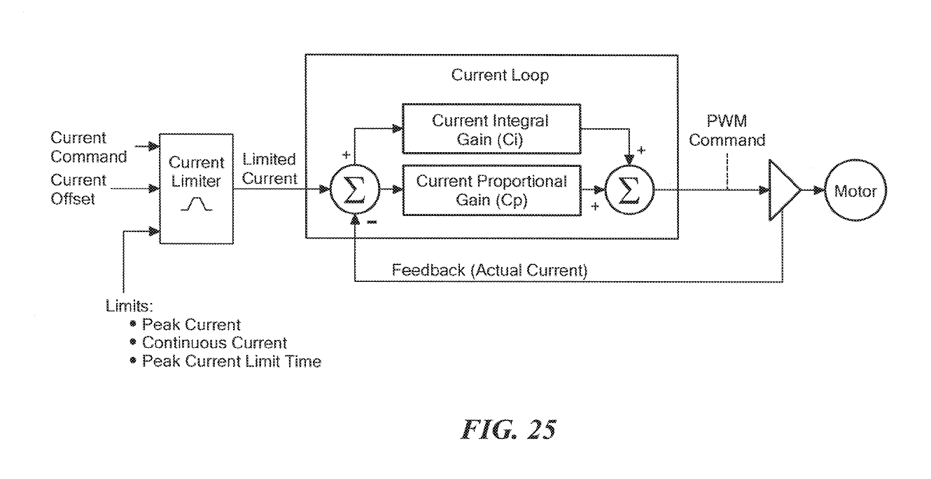

In the field of motion analysis, pelvic obliquity is specified in degrees of angular rotation. To comply with this standard, position feedback is offered to the controller in the same format. The RGR training system uses two linear position measurement units, which are attached to either side of the pelvic brace and operate in the vertical direction. These units are a linear potentiometer and an emulated encoder (internal to the actuator). Position feedback coming from the linear actuator is described above. The pelvic obliquity angle of the pelvic brace is calculated using the relative position of the two attachment points on the pelvic brace (in the vertical direction) and the distance between these two points. Referring to FIG. 24, D is the length of a direct line between the two attachment points, and y is the distance between them in the vertical direction. As one side of the pelvis moves upwardly with respect to the other, the segment of length D spanning the two attachment points rotates. The resulting angle of rotation θ is the pelvic obliquity angle.

To apply impedance control at the obliquity level, the control algorithm from the linear-motion case discussed above can be adapted to act on angular position error measured in degrees of the pelvic obliquity angle. This system's block diagram is presented in FIG. 26, and the details of the PD controller block are shown in FIG. 27. The strength of the force field is specified with the proportional gain K_{c}, with units of N−m/deg. For convenience, the derivative gain B_{c} is not specified independently, but is computed based on the damping ratio, using standard procedures known in the art.

This type of approach to gain selection allows fast changes to be made to the force-field strength while the general dynamic properties of the system remain unchanged. The PD gains produce a force command, which is executed by the impedance controller's force control loop. Referring to FIG. 26, the PD controller acts on the obliquity error and outputs the appropriate force command. Low pass filters 1 and 2 are RC anti-alias filters. FIG. 27 illustrates details of the PD gain block form FIG. 26. The proportional gain K_{c} is specified at the obliquity level, while the derivative gain B_{c} acts on the linear velocity error at the actuator level. B_{c} is computed from K_{c} (the linear motion equivalent) and the specified damping ratio ζ, as would be known in the art. The velocity feedback undergoes secondary filtering (after the velocity error is computed).

Pelvic obliquity reference trajectory is a time series, containing the relationship between space and time. Therefore, in addition to being properly positioned in space, the individual data points of the reference trajectory also have to be presented to the impedance controller at the right time. Therefore, a synchronization algorithm is implemented in the RGR training. One suitable synchronization algorithm is available at D. Aoyagi, W. E. Ichinose, S. J. Harkema, D. J. Reinkensmeyer, and J. E. Bobrow, “A robot and control algorithm that can synchronously assist in naturalistic motion during body-weight-supported gait training following neurologic injury,” IEEE Transactions on Neural Systems and Rehabilitation Engineering, vol. 15, pp. 387-400, 2007 (“the Aoyagi synchronization algorithm”).

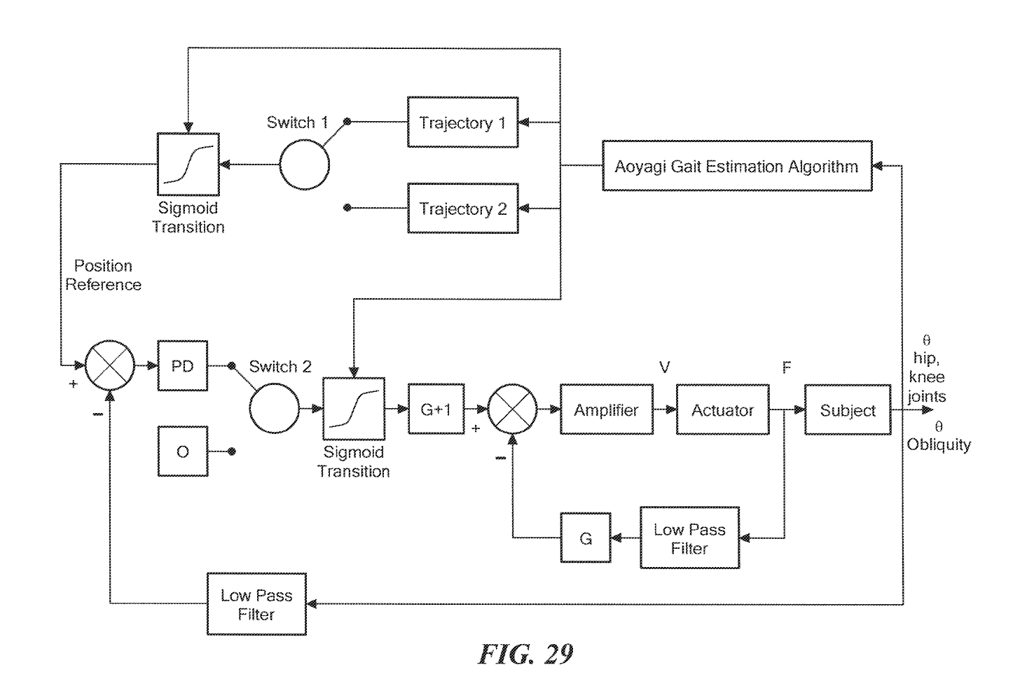

The duration of a single gait cycle spans between two consecutive left heel strikes. The right heel strike occurs at the 50% mark in the gait cycle (assuming symmetrical gait). The Aoyagi synchronization algorithm estimates the actual temporal position of the subject within his gait cycle based on the angular positions and velocities of the subject's hip and knee joints (8 degrees of freedom). A reference for the synchronization algorithm is constructed by recording an 8-dimensional time series over several gait cycles and finding the normalized mean of each DOF. The 8 DOFs are normalized to ensure that they are assigned equal weight. The reference is generated by the norms of the individual vectors, and is represented by the loop of discrete points in FIG. 29. The number of discrete points in the reference is a function of walking cadence and sampling rate used.

During operation, a minimization operation of the norm of the difference between the measured 8-dimensional vector and every vector in the reference is performed, and this identifies the location of the nearest neighbor. This result is normalized to give an index value ranging between 0 and 1. This represents the location of the subject in the temporal sense in the gait cycle.

The human-robot interface features knee and hip angle measurement (4 DOFs). Taking derivatives of these signals produces four angular velocities, for a total of 8 DOFs for use in the Aoyagi synchronization algorithm. In addition, a low profile assembly with a micro switch, which is placed in the subject's left shoe, is used to detect left heel strikes. In one embodiment, the micro switch is mounted on an aluminum sheet sized to fit in the shoe and covered with a plastic sheet for user comfort. Knowledge of such a discrete gait event is useful for both generating synchronization reference trajectories, and for synchronization algorithm performance validation purposes.

Signals from the four rotary potentiometers at the hip and knee joints are analog low pass RC-filtered and sent to the data acquisition card. Heel strike signal, which is also collected, is used to parse the data and find 8 means of the 8 DOFs (hip and knee angular positions and velocities) across the multiple gait cycles.

The overall control system architecture is illustrated in FIG. 29. This control system includes a first controller built up around the pelvic obliquity impedance controller, discussed above, and includes the Aoyagi gait estimation algorithm discussed above. This control system allows for modulation and fine-tuning of the force field applied on to the user in two ways.

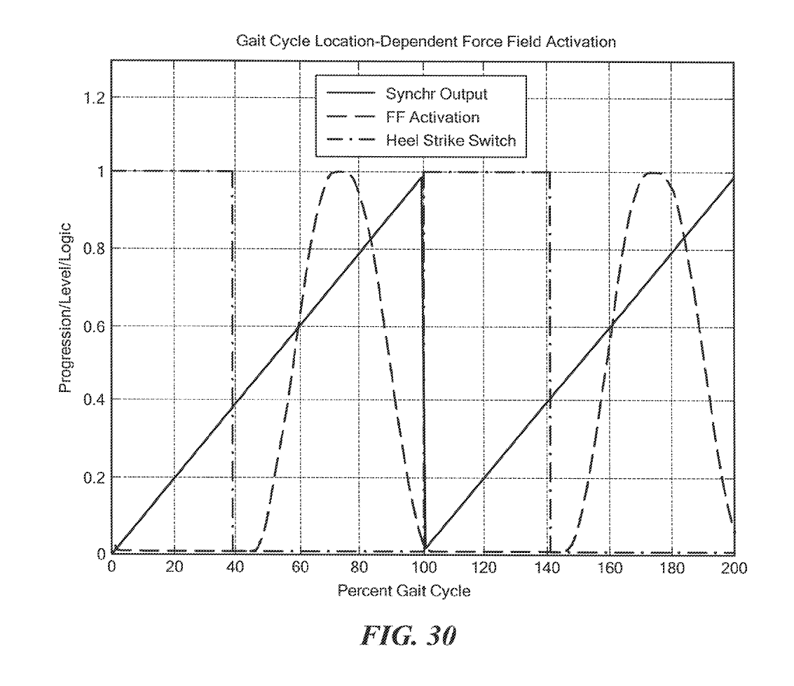

First, the controller can switch between two (or more if necessary) different position references while in operation, within two consecutive gait cycles. The user's hip and knee joint angular positions and velocities are used by the Aoyagi gait estimation algorithm to produce an estimate of the user's point in the gait cycle at any time. This estimation of the point in gait is used in two lookup tables to generate two position references. Switch 1 shown in FIG. 30 executes a transition between the two reference trajectories. This switch follows a sigmoid curve, which is a section of a 3 Hz sinusoid, spanning between 0 and 1. Switch 1 is set to go on or off beginning at 20% of the gait cycle, when the contralateral leg is in stance.

The second way to control the force field applied onto the subject is through precise activation and de-activation of the impedance gains. Switch 2 in FIG. 29 follows a sigmoid curve as well, enabling a smooth transition from the backdrivable mode (zero force control) to impedance control mode, when the PD gains set the desired stiffness and damping (the force field). FIG. 30 illustrates an example of the operation of Switch 2 over to consecutive gait cycles. The synchronization algorithm output predicts left heel strikes and gives a good estimate of gait cycle location mid-stride. The gait estimation (Synchr Output) is the progression through the gait cycle from 0 to 1 (100%). The force field activation sigmoid switch (3 Hz) was set to go on at 44% and off at 76%. Heel strike is marked by the rising edge of the ‘Heel Strike Switch’ signal.

The ability to precisely control the timing of force field activation within the gait cycle only when the contralateral leg (the leg on the hemiparetic side of the body due to stroke) is in swing, means that the moments applied onto the pelvis are not indeterminate, despite the fact that only one actuator is used to apply an external force, as shown in FIG. 7. This allows for adjustments in the PD gains when the force field is in the de-activated state.

In one embodiment, the servo amplifier employs a Schmitt trigger in its enable function to recognize an “enable” signal (for example, greater than 3.65V) and a “disable” signal (for example, less than 1.35 V). Advantageously, any drive signals sent to the actuator via the servo amplifier should be disabled when the control software or the computer fails, as a safety measure. In one embodiment, a safety circuit (shown in FIG. 19) is provided to send an enable signal to the servo amplifier only when the control software is active. A dedicated DAQ output is configured to supply a sinusoidal voltage signal of 100 Hz frequency and ranging between 0 V and 10 V. This signal is routed through an analog RC high-pass filter, with the cutoff frequency on the order of several Hz to avoid excessive signal attenuation. Then, the signal is rectified with a Gratz bridge rectifier and smoothed with help of a capacitor placed in parallel. The result is a slightly varying voltage output which successfully enables the servo amplifier when the input is of proper frequency and magnitude. At the same time, the circuit's output changes to 0 V whenever the input is 0 V or un-varying (as in the case of software error). Using an equation governing discharge of a capacitor:

V_{c}=V₀e^{-t/RC} (4.18)

Solving for time t:

$$t = RC ln ( \frac{{V}_{0}}{{V}_{C}} ) \qquad ( 4.19 )$$

With R=820 kOhm and C1=C2=4.7 μF, the time for the voltage to drop from maximum 10 V to Schmidt trigger's 3.65 V “on” limit is 3.9 s, and dissipation from 5V takes 1.2 s.

Several protocols for use of the robotic gait rehabilitation training system have been developed to assist patients in overcoming the secondary gait deviation of hip hiking and to study the gait of healthy people. The protocols are used to guide the pelvis in the frontal plane via force fields to alter pelvic obliquity and induce motor adaptations in pelvic obliquity control.

Protocol 1

The RGR trainer system, configured to apply vertical forces on the left side of the body, was programmed to switch between two reference trajectories: baseline and hip-hiking. The switching action was designed to happen quickly but smoothly, occurring when the left leg is in stance (due to the small position error at that time) and following a sigmoid curve at a frequency of 3 Hz. The sigmoid is one half cycle of a 3 Hz sinusoid, minimum to maximum amplitude or vice versa, spanning between 0 and 1.

In this protocol, the user walks at a selected walking speed, such as 1.8 km/h, on the treadmill inside the RGR training system and selects a comfortable cadence at this speed. The actuation system operated under zero force control (back-drivable mode), minimizing interaction forces and allowing for maximum freedom of movement.

Baseline pelvic obliquity and hip and knee joint angles are collected over 100 strides and converted into the baseline pelvic obliquity reference trajectory and synchronization reference respectively, by segmenting the data according to heel strikes (as detected by a foot switch in the subject's left shoe) and averaging across all gait cycles.

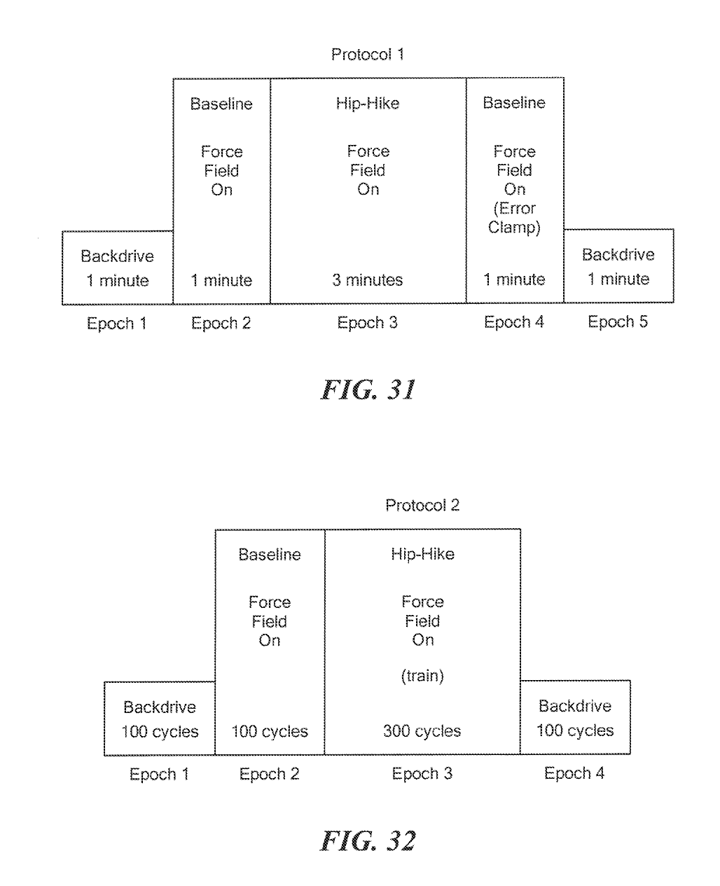

Four time epochs are played out to form a continuous run, as outlined in FIG. 31. Throughout, force, position and gait cycle location data were recorded continuously.

Epoch 1.

The subject walks freely (back-drivable mode) on the treadmill at the specified speed, for a specified time duration, with a metronome setting the cadence. The actuation system is synchronized to the subject's gait by using the subject's own reference synchronization trajectory (8-DOF).

Epoch 2.

The force field is activated, for a specified time duration, with the subject's own baseline still serving as reference trajectory.

Epoch 3. Adaptation Period.

The reference trajectory is switched from baseline to the hip-hiking pattern, for a specified time duration.

Epoch 4. De-Adaptation Period.

The position reference is switched to subject's own baseline, with the force field still active, for a specified time duration.

Epoch 5.

The force field was switched off (backdrivable mode), for a specified time duration.

Protocol 2

Subject walks at his comfortable walking speed (CWS) on the treadmill inside the RGR training system and selects his own cadence at this speed. The actuation system is operated under zero force control (backdrivable mode), minimizing interaction forces and allowing for maximum freedom of movement.

Baseline pelvic obliquity data and hip and knee joint data are collected and converted into a baseline pelvic obliquity reference trajectory and synchronization reference respectively by segmenting the data according to heel strikes (as detected by a foot switch in the subject's left shoe) and averaging across all gait cycles.

The subject walks again at his comfortable walking speed, while performing a simulated hip-hiking gait pattern. A tunnel can be set around a hip-hiking reference trajectory to make the switch between modes less perceivable by the subject. The tunnel can by implemented in the controller by nullifying the position error while it is less than a particular value (tunnel semi-width), and once the position error surpasses the tunnel semi-width, it is offset by that value.

Four epochs are played out to form a continuous run, as outlined in FIG. 32. The epoch durations were based on the number of gait cycles completed, as opposed to the time elapsed as was done in Protocol 1. Throughout the protocol, the interaction force, pelvic obliquity angle and gait cycle location data are recorded continuously.

Epoch 1.

The subject is allowed to walk freely on the treadmill at their previously found CWS, for a specified number of gait cycles, such as 100.

Epoch 2.

With the subject's baseline pelvic obliquity as the position reference, and with the tunnel size set at 1 degree (half-span), the force field is activated, for a specified number of gait cycles, such as 100.

Epoch 3.

The reference trajectory is switched from the subject's own baseline to the hip-hiking trajectory, for specified number of gait cycles, such as 300.

Epoch 4.

The force field is switched off. This epoch differs from that used in Protocol 1, since the subject is not forced to switch back to own baseline (error clamp), but is given freedom to continue walking with the newly-acquired gait pattern. This epoch is used to record the outcome of gait retraining, which occurred in epoch 3.

In Protocol 2, the outcome measure was the degree of hip-hike in the subject's pelvic obliquity immediately following the hip-hike training epoch.

Protocol 3

The protocol is as follows:

The subject walks at his CWS on the treadmill inside the RGR training system and selects his own cadence at this speed. The actuation system operated under zero force control (backdrivable mode), minimizing interaction forces and allowing for maximum freedom of movement.

Baseline pelvic obliquity data and hip and knee joint data are collected by recording the RGR training system's pelvic brace position measurement and hip and knee angle measurements, and converted into the baseline pelvic obliquity reference trajectory and synchronization reference respectively by segmenting the data according to heel strikes (as detected by a foot switch in the subject's left shoe), and averaging across all gait cycles.

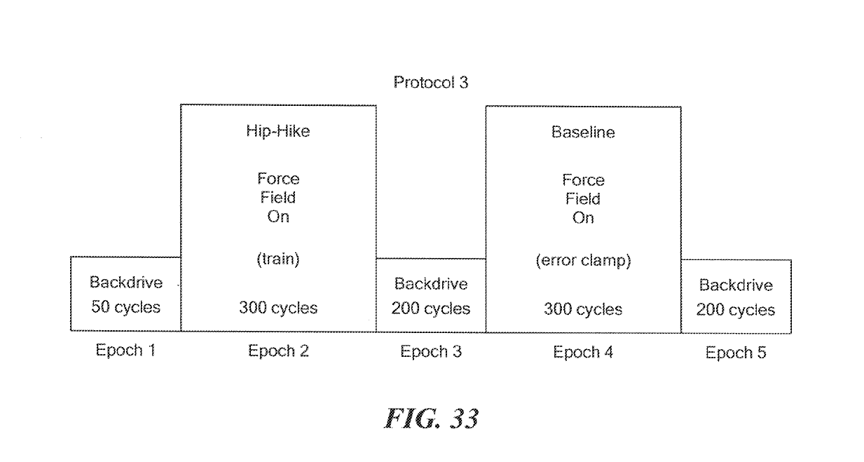

Five different force field levels (for example, K_{c}=20, 25, 30, 35 and 40 N−m/deg) are randomized. Referring to FIG. 33:

Epoch 1.

The subject ambulates, and reaches a steady state pace, for a specified number of gait cycles, such as 50.

Epoch 2.

The subject is exposed to a force field selected randomly out of five different force field levels, with the representative hip-hike pattern serving as the position trajectory, activated between 55% and 85% of the gait cycle, for a specified number of gait cycles, such as 300.

Epoch 3.

A specified number of gait cycles, such as 200, are used to record the outcome of gait retraining.

Epoch 4.

An error-clamp setting, with a tunnel set to +/−0.7 degrees and a force field of K=30 N−m/deg, is used, for a specified number of gait cycles, such as 300.

Epoch 5.

The force field turned is off for a specified number of gait cycles, such as 200. Obliquity from this epoch is used to confirm that de-adaptation is sufficient. One potential method is computing the sample variance s₂² and performing an F-test against reference (baseline) variance.

Epochs 2 through 5 are repeated for the other four levels of force field strength. The timing of force field activation (sigmoid switch) is selected to occur after the initial reversal of the pelvis' direction of motion had occurred (from pelvic drop to hip-hike).

Protocol 4

Protocol 4 compares both assistive and resistive training.

During the assistive training, subjects are instructed to follow the guidance of the RGR training system, and during the resistive training, the subjects are instructed to maintain their own natural gait pattern and not to allow the RGR training system to alter it. For each training type, two variations of epoch 3 were used: ‘backdrive’ and ‘playback’. In the backdrive epoch (epoch 3b), the actuation system operates in backdrivable mode, while in the playback epoch (epoch 3p), the mean commanded force profile from the last ten gait cycles of epoch 2 (the epoch immediately preceding epoch 3p) is played back throughout the duration of epoch 3p. Therefore, while the subjects are exposed to a force field in epoch 2, in epoch 3p they are exposed to a constant force profile, which is only a function of the subject's temporal progression through the gait cycle, and not a function of their pelvic obliquity angle.

Each session can include several, for example, three trials, with each trial testing one of three force field magnitudes (such as 5, 15 and 25 N−m/deg), randomized in order. Each trial lasts a selected number of strides, such as 1200, including four 300-stride epochs: hip-hike train (epoch 2), backdrive (epoch 3b) or playback (epoch 3p), error clamp (epoch 4), and backdrive (epoch 5). In protocol 4 a tunnel around the hip-hiking reference is not used. The force field activation switch was set to go on at 44% of the gait cycle (coinciding very closely with toe-off) and to go off at 76% (in order to diminish to zero by left heel strike—the end of the gait cycle).

The subject walks at his CWS on the treadmill inside the RGR training system and selects his own cadence at this speed. The actuation system operates under zero force control (backdrivable mode). A metronome is set to the subject's cadence.

As the subject ambulates for a specified number of gait cycles, such as 100, to the cadence set by the metronome, baseline pelvic obliquity timeseries and hip and knee joint angle time series are collected and converted into the baseline pelvic obliquity reference trajectory and synchronization reference respectively, by segmenting the data according to heel strikes (as detected by a foot switch in the subject's left shoe), and averaging across all gait cycles.

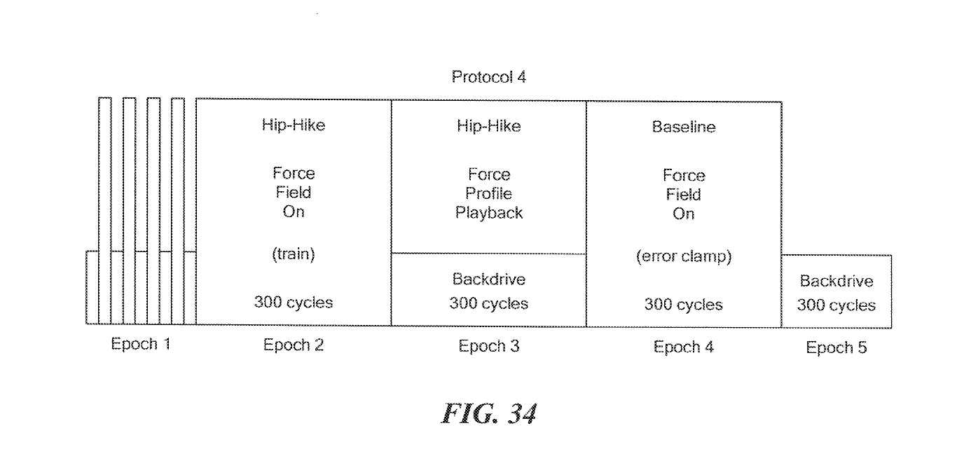

The details of Protocol 4 are as follows, with reference to FIG. 34. With the metronome setting the cadence, the subject ambulates in the RGR training system for a specified time duration, such as 5 minutes, with the system in backdrivable mode in order to reach steady state.

Epoch 1.

Initiation: every 5 strides, the RGR training system switches between two operating modes: error clamp (baseline reference) and hip-hike train. This is done to make the subjects accustomed to the operation of the system, and to make subjects believe that there are only two operating modes. The epoch continues for a specified number of gait cycles, such as 50.

Epoch 2.

The subject is exposed to a force field selected randomly out of three force field levels, with the representative hip-hike pattern serving as the position trajectory, activated between 44% and 76% of the gait cycle, for a specified number of gait cycles, such as 300.

Epoch 3b.

The system is operated in backdrivable mode, for a specified number of gait cycles, such as 300.

Epoch 3p.

The system is operated in playback mode, generating a constant force profile (mean commanded force from last 10 gait cycles in epoch 2) as a function of temporal progression through the gait cycle, for a specified number of gait cycles, such as 300.

Epoch 4.

Error-clamp (K_{c}=15 N−m/deg) with subject's own baseline trajectory is used to de-adapt the subject, for a specified number of gait cycles.

Epoch 5.

The system is operated in backdrive mode, for a specified number of gait cycles, such as 200. Pelvic obliquity during this epoch could be used to confirm de-adaptation.

The RGR training system can incorporate alternative embodiments. For example, besides hip-hiking, another common secondary gait deviation occurring in the motion of the pelvis is circumduction with exaggerated pelvic rotation, as shown in FIG. 5 Guiding pelvic rotation in order to affect this gait deviation requires the ability to generate moments in the horizontal plane. Accordingly, a 2 DOF RGR training system, which can apply moments about pelvic obliquity and pelvic rotation, can be utilized.

The system is able to apply corrective moments to pelvic obliquity and pelvic rotation, while allowing close to free translations in the horizontal plane. As is the case with any impedance-controlled device for human interaction, the inertia of the system should be kept to a minimum, in order to enhance the system's ability to display the prescribed force fields. Considering the specific task at hand, the inertia of the system should be less than that of the actuated body part. The body segment inertias can be found using equations in a NASA publication, based on the total body weight (TBW). Anthropometric source book volume I: Anthropometry for Designers (NASA RP-1024), 1978. Static friction can cause the subject to lose balance. Therefore, the maximum allowable static friction force in the horizontal plane was found to be 8.3 N. That same source found the maximum desirable stiffness applied onto the body to be 4150 N/m.

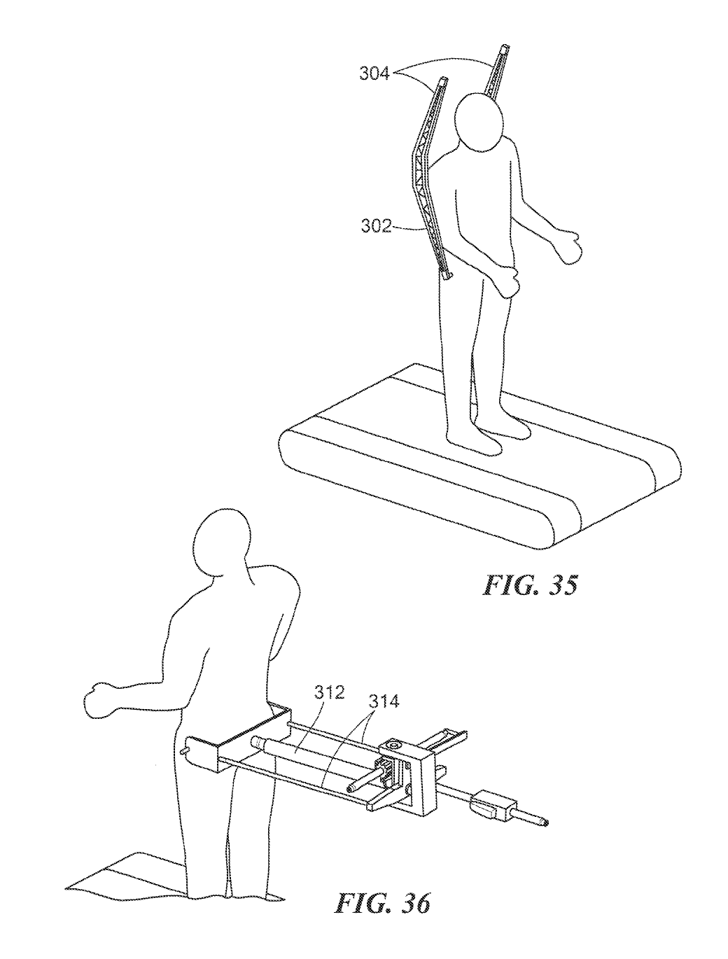

In one embodiment, referring to FIG. 35, two rigid links 302 apply a moment at pelvic obliquity from behind, and two links 304 applying moment at pelvic obliquity from above the patient. In this embodiment, the body weight support system is integrated into the obliquity control parallelogram mechanism. The two linkages must fully support the weight of the subject.

Referring to FIG. 36, an embodiment is illustrated for applying a corrective moment to pelvic obliquity using a shaft 312 in torsion and two push-rods 314 in pelvic rotation, with two linear actuators. Such a shaft should allow for unrestricted motion in the vertical and lateral directions (within certain limits), via two flexible universal joints. Since the torsion bar is of fixed length, as it rotates, the links which apply moments in pelvic rotation must rotate with the torsion bar as well. Therefore, the system has a significant moment of inertia in pelvic obliquity. As this system does not provide body weight support, a separate overhead system can be used to accomplish body weight support.

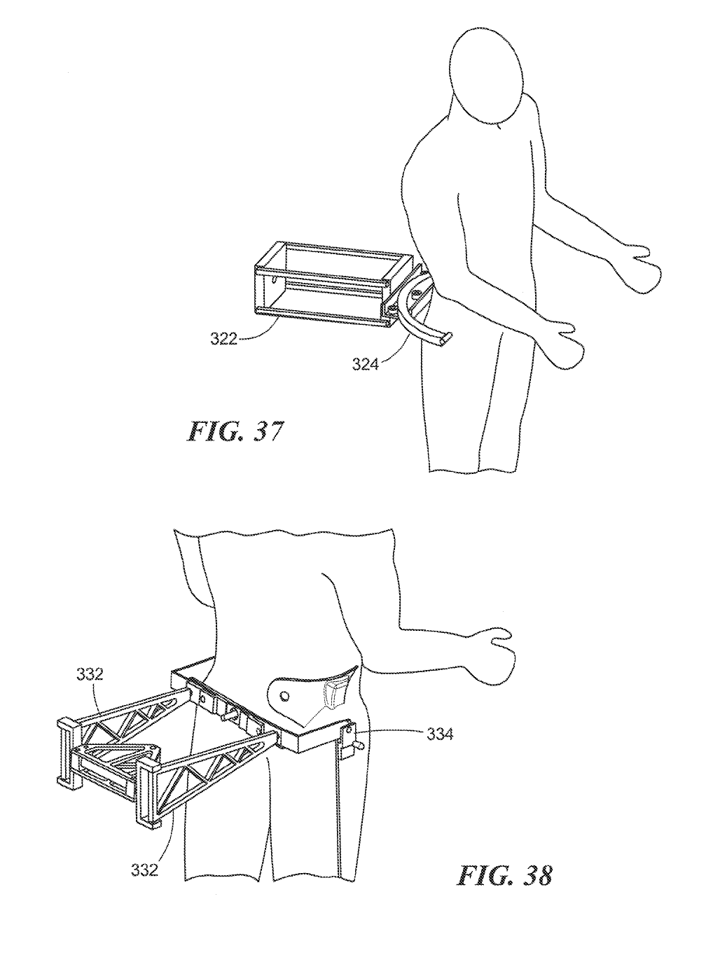

A further embodiment, illustrated in FIG. 37, includes two parallel four-bar mechanisms 322, which support a cylindrical joint for pelvic obliquity, and a semi-circle 324 supported by bearings, which operates at the pelvic rotation level with the remote center of rotation placed inside the subject's body. This embodiment requires flexible transmission to deliver the driving moments to the two rotational DOFs. This reduces the mechanism's inertia (actuators are stationary).

A further embodiment is illustrated in FIG. 38. This embodiment employs two triangular beams 332, which are constrained to rotate together. This creates a structure fixed in rotation but free to translate, for application of the moment to pelvic obliquity. Gimbals 334 at the hip joints allow for hip abduction/adduction and flexion/extension. Flexible transmission is required to apply moments at pelvic obliquity. This embodiment relies on the rigidity of the two triangular arms and the shaft connecting them, in order to apply the prescribed moment to pelvic obliquity. A well designed structure with high stiffness would result in high natural frequency, which is necessary to prevent control issues due to noisy force feedback signal. An offset axis of rotation at the base of the mechanism shifts the center of moment application in pelvic rotation to the inside of the body. This embodiment features significant moment of inertia in the controlled DOF, as well as complexity.

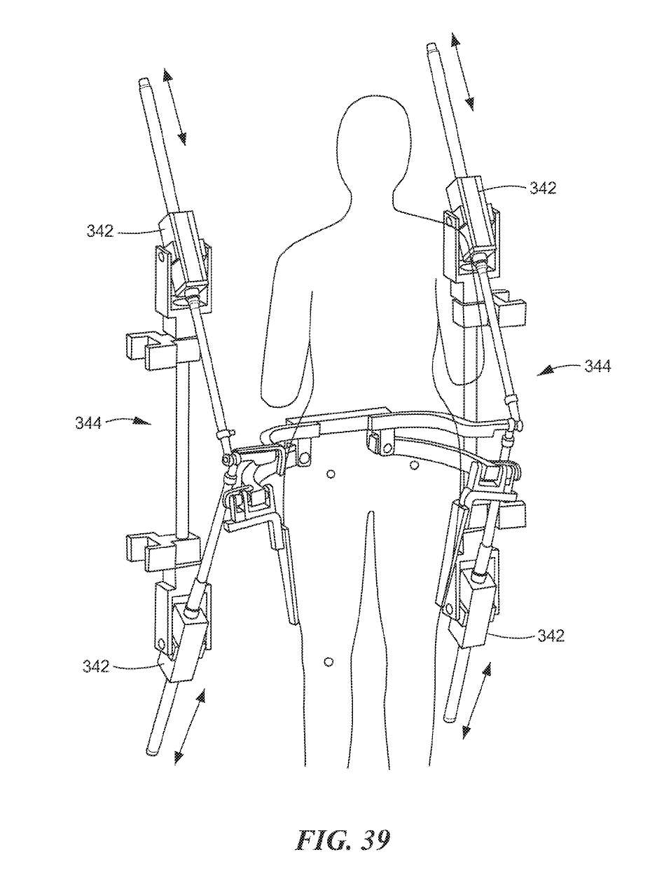

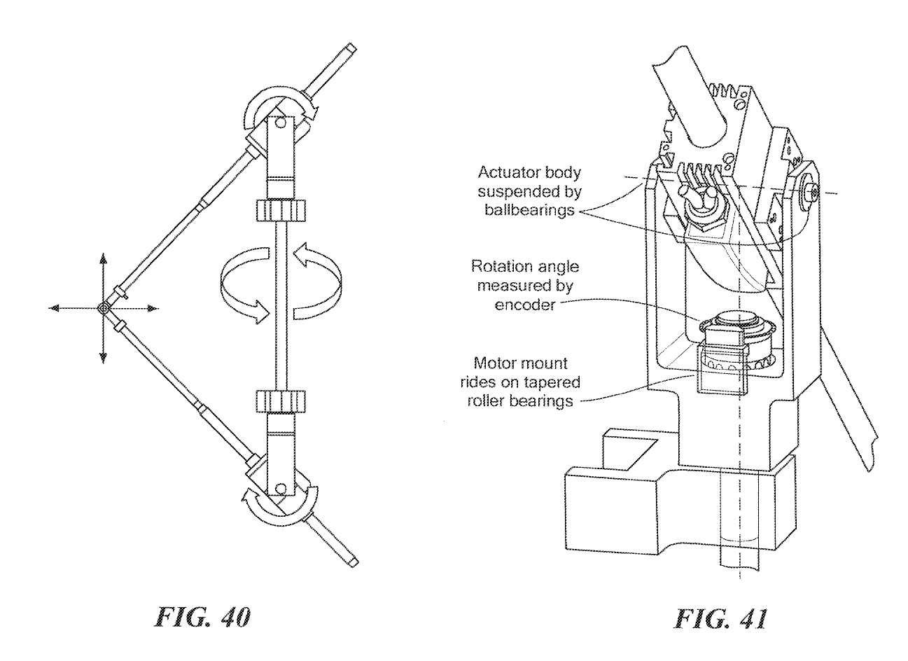

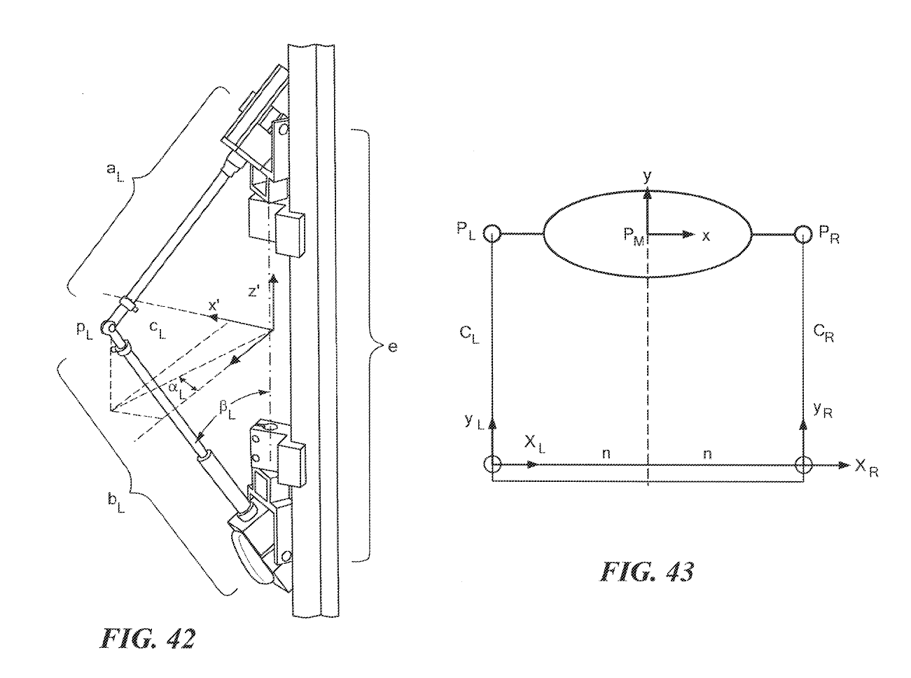

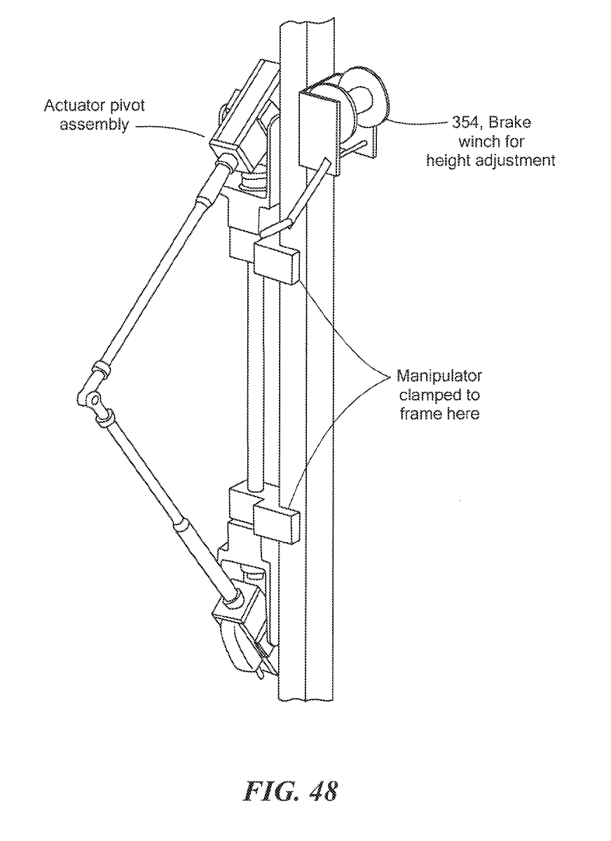

A further embodiment is illustrated in FIGS. 39-50. This RGR training system uses four linear electromagnetic actuators 342 to apply forces in three degrees of freedom: pelvic rotation, pelvic obliquity and vertical translation. The use of direct-drive electromagnetic linear actuators, the housing of which are supported by an immovable frame 350 (FIG. 47) and the moving parts of which are relatively light-weight, leads to good interaction force modulation.

The system applies force fields in pelvic obliquity, pelvic rotation, and the vertical direction, that is, in three degrees of freedom (DOF). The remaining DOFs are left free. Allowing patients to execute their natural patterns of pelvis translation leads to a feeling of a more natural walk and better control of balance, thus leading to better results for the patients.

More particularly, the RGR training system includes two planar manipulators 344 to apply forces to the right and left sides of the pelvic brace. Each manipulator includes two linear actuators 342. Working in unison, the two manipulators can apply forces (in the vertical direction), moments (applicable to pelvic obliquity and pelvic rotation), or both onto a pelvic brace worn by the patient. In general, this mechanism cannot apply forces in the transverse direction (side to side). As a result, horizontal translations are not actuated. The mechanism can actively respond to the environment's force input under zero-force-control, in order to minimize the interaction force. Thus, the mechanism is back-drivable in the actuated DOFs. Each planar manipulator provides mounting for two linear actuators, which pivot about their center of housing in order to minimize their moments of inertia. The linear actuators are suspended on ball bearings to reduce friction.

The following equations describe kinematics of the left-hand side closed link mechanism. The angle β_{L} is found from the following equation:

$${β}_{L} = arccos ( \frac{{b}_{L}^{2} + {e}^{2} - {a}_{L}^{2}}{2 * {b}_{L} * e} )$$

Now using β, the distance from the endpoint to the vertical axis can be found:

c_{L}=b_{L} sin β_{L}

The angle of rotation is measured directly (α), so that vector p can be described, which locates the mechanism's endpoint with respect to the origin xyz.

$${p}_{L}^{′} = [ \begin{matrix}c * sin α \\ c * cos α \\ b * cos β\end{matrix} ]$$

The position of the endpoint on the right-hand side is found in the same exact way, giving two vectors, which describe the locations of P′_{L} and P′_{R} with respect to the two origins located on either side of the device. Next, the locations of these two points are found with respect to the default location of the subject in the system, as shown in FIG. 43.

Now we employ translation in order to find the position vectors of points P_{L} and P_{R} with respect to the main reference frame (xyz):

$${P}_{L} = {P}_{L}^{′} + [ \begin{matrix}- n \\ - {C}_{L} \\ 0\end{matrix} ]$$

$${P}_{R} = {P}_{R}^{′} + [ \begin{matrix}n \\ - {C}_{R} \\ 0\end{matrix} ]$$

The pelvic rotation θ and pelvic obliquity φ angles are found using the above position vectors:

$${P}_{R} = {P}_{R}^{′} + [ \begin{matrix}n \\ - {C}_{R} \\ 0\end{matrix} ]$$

$$φ = arctan ( \frac{{P}_{L ( z )} - {P}_{R ( z )}}{\sqrt{{\{ {P}_{R ( z )} - {P}_{L ( z )} \}}^{2} + {\{ {P}_{R ( y )} - {P}_{L ( y )} \}}^{2}}} )$$

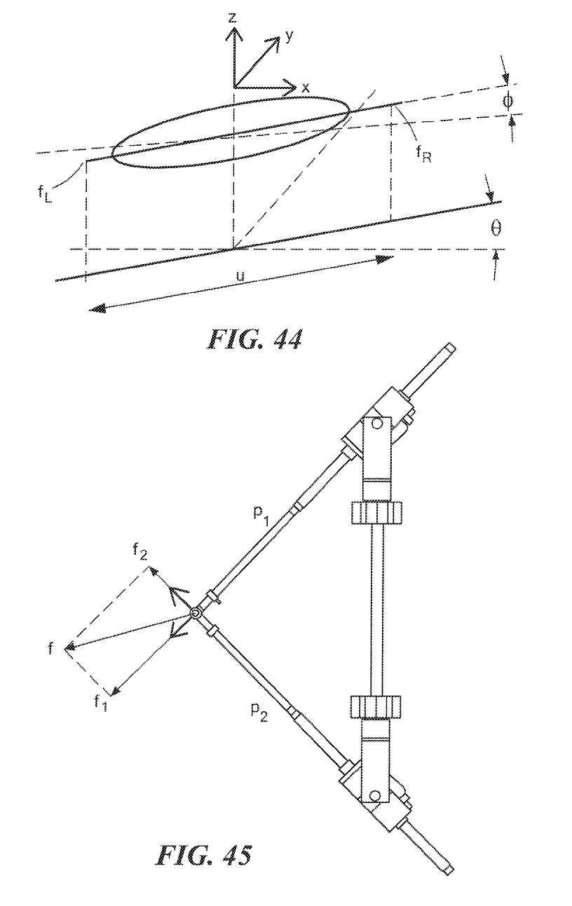

FIG. 44 provides a definition of pelvic obliquity Φ and pelvic rotation θ angles. The pelvic brace is viewed from behind.

The two force vectors necessary to impart the desired moments T_{θ} and T_{φ} onto the pelvis are found as follows:

$$φ = arctan ( \frac{{P}_{L ( z )} - {P}_{R ( z )}}{\sqrt{{\{ {P}_{R ( z )} - {P}_{L ( z )} \}}^{2} + {\{ {P}_{R ( y )} - {P}_{L ( y )} \}}^{2}}} )$$

$${f}_{R} = \frac{1}{2} \{ \begin{matrix}0 \\ {T}_{z}\end{matrix} \} - \frac{1}{u} \{ \frac{- cos θ {T}_{θ} + sin θ sin φ {T}_{φ}}{cos φ {T}_{φ}} \}$$

FIG. 44 also illustrates the forces f_{L} and f_{R} necessary to produce desired net forces and moments.

$$f = [ \begin{matrix}{v}_{1} & {v}_{2}\end{matrix} ] \{ \frac{\vert {f}_{1} \vert}{\vert {f}_{2} \vert} \}$$

Now substitute:

$${v}_{i} = \frac{{p}_{i}}{\vert {p}_{i} \vert}$$

and solve above equation for |fᵢ|.

$$\{ \begin{matrix}\vert {f}_{1} \vert \\ \vert {f}_{2} \vert\end{matrix} \} = {[ \begin{matrix}\frac{{p}_{2}}{\vert {p}_{1} \vert} & \frac{{p}_{2}}{\vert {p}_{2} \vert}\end{matrix} ]}^{- 1} f$$

Thus the magnitudes of the force commands are obtained, as shown in the equation above, which should be sent to the two actuators of a planar manipulator in order to produce the required force f, as is shown in FIG. 45. The forces from the two manipulators (f_{R} and f_{L}) produce the required torques, which are applied onto the pelvis through the pelvic interface.

This embodiment expands the functionality of the RGR training system, by providing the ability to apply corrective torques to pelvic rotation. Abduction combined with exaggerated pelvic rotation is the second most common secondary gait deviation in control of the pelvis, after hip-hiking, and therefore in general it is desirable to be able to address this particular gait deviation in the future.

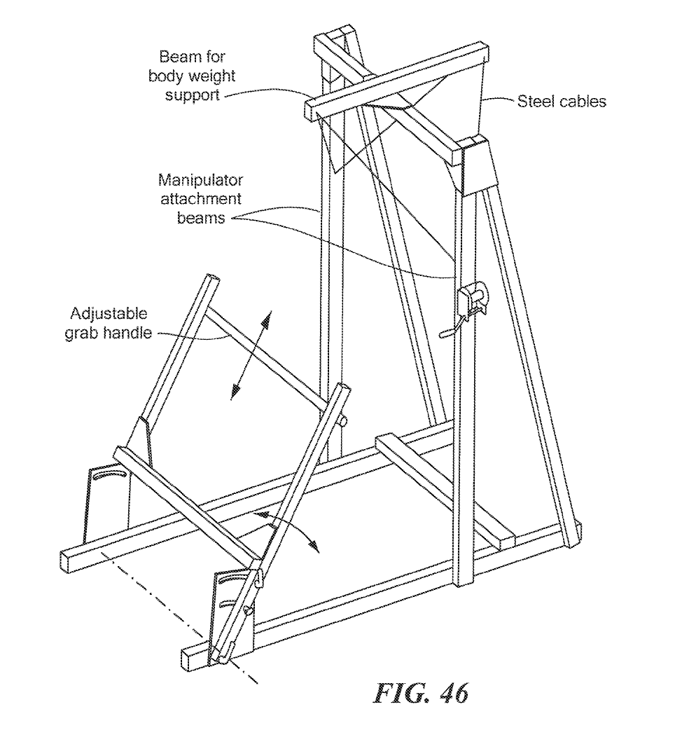

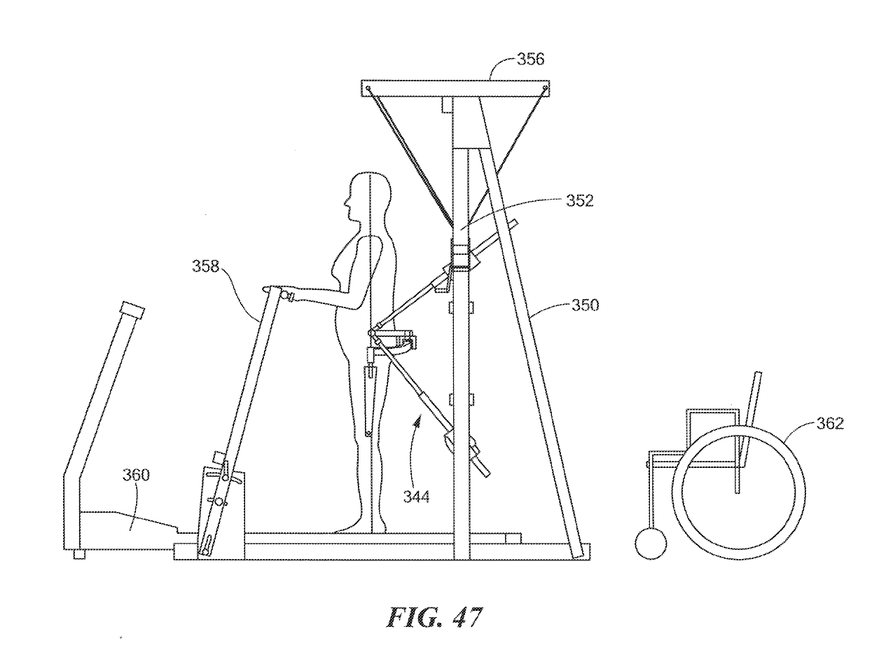

The planar manipulators 344 are mounted to a frame 350, illustrated in FIGS. 46-48. The frame provides a rigid support for the linear actuation system. Each planar manipulator is attached to the frame by, for example, clamping to an upright structural element 352. A brake winch assembly 354 is provided to adjust the height of the planar manipulator 344 on the frame. The frame fits over a treadmill 360 and is sufficiently wide at the rear to allow a patient to transfer to the treadmill from a wheel chair 362. An overhead beam 356 with steel cables can provide additional body weight support.

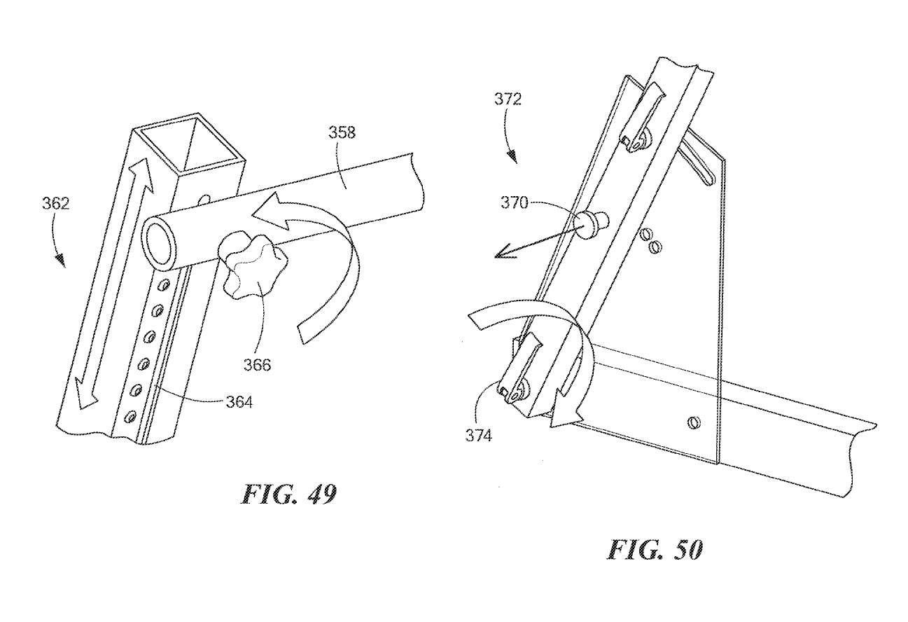

The frame can include a handlebar 358 for the patient to grasp while using the system. The handlebar can include a height adjustment mechanism 362. One embodiment of a height adjustment mechanism, illustrated in FIG. 49, includes a index device 364 and tightenable knob 366. The handlebar can also include a fore and aft adjustment mechanism 372, such as an angular or tilt adjustment mechanism. One embodiment of a tilt adjustment mechanism, illustrated in FIG. 50, includes quick-release clamps 374 and a spring loaded plunger 376.

The present system incorporates the advantageous qualities of high back drivability and force controllability, with impedance control. The control system is able to modulate the forces applied onto the body depending on the patient's efforts. The system allows all of the natural motions of the pelvis and features a lower body exoskeleton that employs the waist, thighs, shanks, and feet to transfer moments to the pelvis. The system, incorporating the lower body exoskeleton, highly backdrivable linear actuator, impedance control and a gait synchronization algorithm, produce a gait retraining system that can effectively and reliably apply corrective moments to pelvic obliquity. The actuation system and human-robot interface of the trainer are simple, with low moving mass and low friction, easing the task of the control system in generating appropriate performance of the overall system. The present system leaves translation in the horizontal plane un-actuated and as friction-free as possible, leading to improved gait

It will be appreciated that components used in the system can be analog or digital. For example, the linear potentiometer described above can be replace with a digital linear encoder to eliminate noise inherent to analog devices. The control system can be implemented in any suitable manner, as can be appreciated by those of skill in the art. Also, those of skill in the art will recognize that various features described in conjunction with one embodiment can be used in conjunction with other embodiments.

The RGR training system can also operate in conjunction with a powered knee orthotic device. This combination can be used to administer gait rehabilitation therapy by addressing both primary and secondary gait deviations, exhibited in the knee joint and the pelvic motion respectively. For example, a powered knee orthotic device can be worn on the affected side, preventing stiff-legged gait, which is the primary gait deviation, which in turn leads to hip-hiking, a secondary gait deviation in the pelvic motion.

The system can also be used for studying gait in healthy people, which may lead to developing better gain retraining therapies for post-stroke patients.

The invention is not to be limited by what has been particularly shown and described, except as indicated by the appended claims.

What is claimed is:

1. A robotic gait rehabilitation training system, comprising:
a frame;
a pelvic brace attachable to a pelvis of a user;
an actuation system including a linear actuator operative to provide a linear force, the actuation system mounted to the frame and pivotably attached at one end to the pelvic brace to transfer forces between the linear actuator and the pelvic brace at a location to provide a moment arm onto the pelvis of the user in a frontal plane to counter pelvic obliquity of the user's hip.

2. The system of claim 1, wherein the actuation system is mounted to the frame and the pelvic brace with joints to follow horizontal motion of the patient.

3. The system of claim 1, wherein the actuation system is pivotably attached to the pelvic brace with a spherical joint.

4. The system of claim 1, wherein the actuation system is rotatably attached to the frame.

5. The system of claim 1, wherein the actuation assembly is mounted to the frame with a mounting assembly comprising a prismatic joint guide for horizontal motion and a revolute joint for rotation about a vertical axis.

6. The system of claim 1, further comprising a pair of leg braces, each attached to the pelvic brace with a movable hip joint and configured to attach to the user's legs at multiple locations.

7. The system of claim 1, wherein the actuation system is backdrivable to modulate forces applied by the linear actuator.

8. The system of claim 1, further comprising a control system in communication with the actuation system to drive the linear actuator.

9. The system of claim 8, further comprising a load cell disposed in linear alignment with the linear actuator to provide feedback to the control system.

10. The system of claim 8, further comprising a linear potentiometer mounted to the frame and to the pelvic brace to provide feedback to the control system.

11. The system of claim 8, wherein the control system is operative to control the actuation system in synchronization with the user's gait.

12. The system of claim 8, wherein the control system is operative to control the actuation system to apply a force when the user's leg is in a swing phase.

13. The system of claim 1, wherein the frame in configured to fit over a treadmill.

14. The system of claim 1, wherein the frame includes a handlebar for grasping by the user.

15. The system of claim 1, further comprising a planar manipulator mounted to the frame and comprising two linear actuators arranged in a plane and meeting at a spherical joint connected to the pelvic brace, and configured to apply moments to counter pelvic obliquity and pelvic rotation in a horizontal plane.

16. The system of claim 15, further comprising a second planar manipulator mounted to the frame on an opposite side.

17. An exoskeleton comprising:
a pelvic brace attachable to a pelvis of a user comprising a shell that wraps around and fastens to the user's waist and a frame assembly attached to the shell; and
a pair of leg braces attached to the pelvic brace with hip joints, each leg brace attachable to the leg at multiple locations extending from the ankle to the thigh, each leg brace including a knee joint.

18. The exoskeleton of claim 17, wherein each of the leg braces is attached to the frame assembly of the pelvic brace with a pair of rotational joints that together define a remote center of rotation coincident with the user's hip joint.

19. The exoskeleton of claim 17, wherein each of the leg braces is attached to the frame assembly of the pelvic brace with a joint to provide internal and external rotation of the hip.

20. The exoskeleton of claim 17, wherein each leg brace includes a thigh component attachable to the user's thigh and a shank component attachable to the user's shank.

21. The exoskeleton of claim 20, wherein the length of the thigh component is adjustable and the length of the shank component is adjustable.

22. The exoskeleton of claim 17, wherein the pelvic brace is adjustable to accommodate hips of different widths.

23. The exoskeleton of claim 17, wherein the angle of the knee joint in the frontal plane is adjustable.

24. The exoskeleton of claim 17, wherein the frame assembly of the pelvic brace includes a back center piece and two side sections, each side section including an upper arm and a lower abductor, wherein the shell attaches to the upper arm of each side section, and the leg braces attached to the abductors.

25. The exoskeleton of claim 17, further comprising one or more angular displacement sensors disposed within the hip joints for communication with a control system to measure a user's gait.

26. The exoskeleton of claim 17, further comprising one or more angular displacement sensors disposed within the knee joints for communication with a control system to measure a user's gait.

27. The exoskeleton of claim 17, further comprising a foot switch disposed on one of the leg braces for communication with a control system.

28. A control system for a robotic gait rehabilitation training system comprising:
a robotic gait rehabilitation training system comprising an actuation system including a linear actuator operative to provide a linear force, the actuation system mounted to a frame and pivotably attached at one end to a pelvic brace attachable to the pelvis of a user to transfer forces between the linear actuator and the pelvic brace at a location to provide a moment arm onto the pelvis of the user in a frontal plane to counter pelvic obliquity of the user's hip;
an impedance controller in communication with the actuation system to receive feedback data from the user and drive the actuation system, the feedback data including pelvic obliquity;
a gait controller in communication with the training system to received hip and knee joint rotation data and operative to estimate a gait cycle of the user from the hip and knee joint rotation data; and
a first controller in communication with the impedance controller and the gait controller and operative to drive the linear actuator in synchronization with the user's gait cycle.

29. The control system of claim 28, wherein the first controller is operative to transition from a fully backdrivable mode with no force control of the actuation system to a impedance control mode with force control of the actuation system.

30. The control system of claim 28, wherein the first controller is operative to transition between modes in synchronization with the user's gait cycle.

31. The control system of claim 28, wherein the first controller is operative to drive the linear actuator during a leg swing phase of the user.

32. The control system of claim 28, wherein the gait controller is operative to determine one or more reference gait cycle trajectories of the user and to estimate any point in the user's gait cycle within a reference trajectory.

33. The control system of claim 32, wherein the reference gait cycle trajectory includes at least one of a baseline gait cycle and a hip-hiking gait cycle.

34. The control system of claim 32, wherein the first controller is operative to switch between two or more reference trajectories.

35. The control system of claim 32, wherein the first controller is operative to switch between the two or more reference trajectories following a sigmoid curve.

36. The control system of claim 28, further comprising a user interface in communication with the first controller.

37. A method of using the robotic gait rehabilitation training system, comprising:
providing the robotic gait rehabilitation training system of claim 1 and a treadmill;
determining a reference gait cycle of a user walking on a treadmill wearing the pelvic brace;
driving the actuation system for at least a portion of the time the user is walking on the treadmill.

38. The method of claim 37, further comprising synchronizing the system to the user's gait while the user walks freely on the treadmill.

39. The method of claim 38, wherein the synchronizing step includes a step of determining a user's baseline gait cycle.

40. The method of claim 38, wherein the synchronizing step includes a step of determining a user's hip-hiking gait cycle.

41. The method of claim 37, further comprising driving the actuation system after allowing the user to walk freely on the treadmill.

42. The method of claim 37, further comprising switching between driving the actuation system and allowing a user to walk freely on the treadmill.

43. The method of claim 37, further comprising driving the actuation system while referencing a user's baseline gait cycle.

44. The method of claim 37, further comprising driving the actuation system while referencing a user's hip-hiking gait cycle.

45. The method of claim 37, further comprising driving the actuation system while switching between a user's hip-hiking gait cycle to a user's baseline gait cycle.

46. The method of claim 37, wherein the actuation system is driven at a constant force.

47. The method of claim 37, wherein the actuation system is driven for a specified time duration.

48. The method of claim 37, wherein the actuation system is driven for a specified number of gait cycles.
